SEQUENCE LISTING (12 sequences)

SEQ ID NO: 1 - protein, 14 aa, Artificial Sequence
  Test Substrate Sequence
CEEEFIYGAFKKKK

SEQ ID NO: 2 - protein, 14 aa, Artificial Sequence
  Test Substrate Sequence
CEEEFIYGAFKKKK

SEQ ID NO: 3 - protein, 13 aa, Artificial Sequence
  Test Substrate Sequence
CEEFIYGAFKKKK

SEQ ID NO: 4 - protein, 13 aa, Artificial Sequence
  Test Substrate Sequence
CEEFIYGAFRRRR

SEQ ID NO: 5 - protein, 13 aa, Artificial Sequence
  Test Substrate Sequence
CEEFIYGAFKKKK

SEQ ID NO: 6 - protein, 7 aa, Artificial Sequence
  Test Substrate Sequence
FIYGAFK

SEQ ID NO: 7 - protein, 10 aa, Artificial Sequence
  Test Substrate Sequence
CAAFIYGAFK

SEQ ID NO: 8 - protein, 14 aa, Artificial Sequence
  Test Substrate Sequence
CEEEFIYGAFKKKK

SEQ ID NO: 9 - protein, 13 aa, Artificial Sequence
  Test Substrate Sequence
CEEFIYGAFKKKK

SEQ ID NO: 10 - protein, 7 aa, Artificial Sequence
  Test Substrate Sequence
LRRASLG

SEQ ID NO: 11 - protein, 7 aa, Artificial Sequence
  Test Substrate Sequence
LRRASLG

SEQ ID NO: 12 - protein, 13 aa, Artificial Sequence
  Test Substrate Sequence
CEEFIYGAFRRRR

(19) United States
(12) Patent Application Publication
(10) Pub. No.: US 2002/0123068 A1
(43) Pub. Date: Sep. 5, 2002

(54) Water-soluble, fluorescent, & electrophoretically mobile peptidic substrates for enzymatic reactions and methods for their use in high-throughput screening assays

(76) Inventors: Brian P. Dwyer, San Diego, CA (US); John R. Havens, San Diego, CA (US)

Correspondence Address: LYON & LYON LLP, 633 WEST FIFTH STREET, SUITE 4700, LOS ANGELES, CA, 90071, US

(21) Appl. No.: 09/775,840

(22) Filed: Jan. 31, 2001

(51) Int. Cl. G01N 33/53; C07K 9/00; C07K 7/08; C07K 7/06; C07K 14/00

(52) U.S. Cl. 435/7.1; 530/322; 530/324; 530/326; 530/327; 530/328

(57) ABSTRACT

The present invention provides non-radioactively labeled synthetic substrates for enzymatic reactions which exhibit markedly improved solubility having the general structure *F-R₁-L₁-R₂-P_{Hc1}-P_{S}-P_{Hc2}-(R₃-L-R₄-T)_{y}. These substrates may be designed to carry a charge to allow electrophoretic separation of substrates and reaction products. The invention also provides enzymatic activity assays for protein kinases, phosphatases and proteases utilizing the substrates of the invention, as well as methods of producing these substrates. In addition, the invention also provides libraries of the substrates, and methods of utilizing these libraries to select optimal synthetic peptide enzyme substrates for high-throughput screening assays.

### FIELD OF INVENTION

The present invention provides labeled synthetic substrates for enzymatic reactions that exhibit markedly improved solubility having the general structure *F-R₁-L₁-R₂-P_{Hc1}-P_{S}-P_{Hc2}-(R₃-L-R₄-T)_{y}. These substrates may be designed to carry a charge to allow electrophoretic separation of substrates and reaction products. The invention also provides enzymatic activity assays for protein kinases, phosphatases and proteases utilizing the substrates of the invention, as well as methods of producing these substrates. In addition, the invention also provides libraries of the substrates, and methods of utilizing these libraries to select optimal synthetic peptide enzyme substrates for high-throughput screening assays.

### BACKGROUND OF THE INVENTION

Protein kinases are a diverse family of enzymes that phosphorylate serine, threonine, or tyrosine residues present in the sequences of protein substrates. The human genome contains approximately 2,000 protein kinases that are potential targets of drug-screening programs. Central to this research are kinase-activity assays in which a wide library of chemical compounds are assayed for their ability to inhibit or activate the kinase reaction in high-throughput screening (HTS) assays. However, one major hurdle in developing an HTS assay for a given protein kinase is identifying a suitable substrate. Peptidic substrates are the most desirable because they are easy to make and purify in large quantity, conjugation chemistries for peptides are well known, and peptide products may be easily separated from substrates by chromatography or electrophoresis.

Libraries of potential peptidic substrates can be easily synthesized using solid-phase peptide synthesis, and they offer the advantage in that many potential substrates can be screened at one time, thereby increasing the odds of finding active candidate sequences. However, a continuing challenge in the art is identifying the few specific active substrates that are vastly outnumbered by inactive members of the peptide library.

Till et al. (1994) J. Biol.Chem. 269, 7423-7428, describes an early attempt to design peptidic substrates for a protein kinase utilizing a peptide library. Peptide libraries had been used to identify protease substrates prior to Till, as referenced therein. The two libraries described in Till each contained only one degenerate position in a sequence of 7 or 13 residues. Mass spectrometry was used to identify the phosphorylated products. Further development in the art of peptide design is shown in Songyang et al. (1994) Current Biology 4, 973-982. Songyang described a process wherein a degenerate peptide library was constructed by solid-phase synthesis in which a phosphate acceptor, in this case tyrosine, was flanked by degenerate positions that could be one of a number of chosen amino acids. The peptide mixture was treated with the protein kinase of interest and γ-[³²P]-ATP. The kinase-treated peptide mixture was then submitted to metal-chelate chromatography to isolate the phosphorylated peptides from the mixture. The isolated peptides were then submitted to sequencing to identify the predominate amino acids at each position and obtain a rough consensus substrate sequence.

Lam et al. (1995) Int. J. Protein Peptide Res. 45, 587-592. Lam et al. (1998) Life Sciences 62, 1577-1583; and Lou et al. (1996) Bioorganic Medicinal Chemistry 4, 677-682 described the use of the Selectide™ process in identifying peptide substrates for protein kinases. In Lam, a random peptide library was produced that contains millions of peptide species on polymer beads, with any given bead containing a single peptide entity. The peptide beads were treated with a protein kinase and γ[³²P] adenosine triphosphate (ATP) and then washed. The washed beads were mixed with hot agarose and spread out on a glass plate. After exposure to x-ray film, the radioactive beads in the gel were identified, collected, and submitted to protein sequencing. Using this process, a peptide substrate for SRC kinase was identified. This substrate proved to be a better substrate than the substrate peptide derived from a natural SRC kinase substrate cdc2. Lou further examined this sequence by making a directed library that contained a IY motif, and repeating the Lam procedure. The next generation peptide identified in this secondary screen was phosphorylated by SRC Kinase twofold greater than the originally identified peptide.

After an appropriate substrate is identified, assays for many protein kinases can be performed using synthetic peptide substrates that contain the recognition sequence of the particular protein kinase of interest with the serine, threonine, or tyrosine residue that is the phosphate acceptor. Traditionally, the detection of the phosphorylated peptidic substrates has involved capture on a phosphocelluose paper filter followed by liquid scintillation counting of ³²P that is incorporated into the peptidic substrate by the action of a specific protein kinase and γ-[³²P]-(ATP). However, radioactive labels have many drawbacks especially related to health risks and disposal requirements. In addition, the expensive radiolabeled reagents have a very short shelf-life on the order of weeks. Because of these difficulties, other bioassay areas such as nucleic acid sequencing or antibody assays have abandoned radioactive labeling for fluorescent labels or other non-radioactive alternative. As a result many versatile high-throughput assay systems originally designed for these areas that would be useful for enzyme inhibitor/activator screens are not compatible for use with radioactive labels.

Alternatively, a fluorescent moiety can be conjugated to the peptidic substrate in order to provide a highly sensitive means of detecting the phosphorylated peptidic substrate after separation from the unphosphorylated peptidic substrate. This separation can be accomplished by chromatographic or electrophoretic means. If electrophoretic means are used, such as described in W. S. Wu, et. al, Analytical Biochemistry, 269: 423-425 (1999), then it is desirable that the substrate for the kinase assay be of a different charge (i.e. positive) than the phosphorylated product. For instance, Lutz et al. (1994) Analytical Biochemistry 220: 268-274, describes the use of electrophoresis for performing a kinase or phosphatase assay using fluorescent substrates. The peptide substrates were labeled with fluorescamine, and the separation is performed in an agarose gel. Although these assays are safer and easier to use than radioactive assays, a primary difficulty in using fluorescently labeled peptides for protein kinase assays is that fluorophores are typically very hydrophobic molecules that contain polycyclic aromatic systems, and are thus sparingly water-soluble. Conjugation of a fluorophore to a peptide can make the peptide much more hydrophobic. As aqueous buffers are required for protein-kinase assays, solubility considerations have greatly limited the range of fluorophores and peptides that may be used in such modified peptide systems.

Thus, it is desirable to use fluorescent substrates and detection methods rather than radioactive substrates and detection methods in a wide range of kinase enzyme assays for activity. However, it is laborious to design peptide substrates for each specific kinase reaction which are both detectably labeled with fluorescent markers, and which are sufficiently hydrophilic for use in most kinase reactions. Thus, a need exists for a method of generally modifying potential peptide substrates to prepare a wide variety of peptidic, fluorescent substrates that are water-soluble and that can be easily separated from their phosphorylated counterparts. Furthermore, simple methods are needed that allow the selection of appropriate peptide substrates for high-throughput enzyme activity assays for uncharacterized enzymes. Because the indigenous substrates for these enzymes is often unknown, methods which do not involve the careful engineering of substrate peptides based on the sequences of their indigenous substrates are needed. The present invention solves these problems in the art by providing uniformly soluble and detectable peptide substrates which can be produced as random, partially random, or weighted random peptide substrate libraries. These libraries of labeled, assay-ready compounds may then be used directly for screening with various enzymes. Thus, fully functional, detectable peptide substrates may be identified without the need for further modification for detection or use in HTS drug development assays. Once identified by the screening methods of the invention, the peptidic substrates may be easily manufactured en masse according to the methods of the production methods described herein.

### SUMMARY OF INVENTION

Thus, in a primary aspect, the present invention is drawn to modified synthetic peptide substrate molecules which are suitable for use in a variety of enzymatic activity assays, including protein kinase assays. The modified synthetic peptide substrates (“substrates”) of the invention have the general formula:

*F-R₁-L₁-R₂-P_{Hc1}-P_{S}-P_{Hc2}-(R₃-L₂-R₄-T)_{y}

wherein *F is a detectable moiety with a molecular weight of less than 5 kD, preferably a fluorescent moiety, a hapten moiety, a chromogenic moiety, or a chemiluminescent moiety, and most preferably a fluorescent moiety;

R₁, R₂, R₃, and R₄ are each, independently: a covalent bond or a covalent linkage consisting of a branched or unbranched, substituted or unsubstituted, saturated or unsaturated chain of 1-10 carbon atoms; 0-3 heteroatoms selected from the group consisting of oxygen, nitrogen, and sulfur; and further consisting of at least one linkage chosen from the group consisting of ether, ester, hydrazone, amide, thioether, thioester, thiourea, disulfide and sulfonamide linkages;

L₁ and L₂ are each, independently: a branched or unbranched hydrophilic uncharged polymer selected from the group consisting of polyethylene glycol (PEG) and polysaccharides, having a molecular weight of about 80 to about 4000 Daltons, more preferably from about 100 to about 2000 Daltons, more preferably from about 500 to about 1500 Daltons;

P_{Hc1} is peptide with the general fonnula A_{c}(A_{H})ₙAₘ,

wherein A_{c} is selected from the group consisting of a covalent bond, ornithine, cysteine, homocysteine, cysteic acid, and lysine;

each of A_{H} is, independently, a charged or uncharged hydrophilic amino acid selected form the group consisting of serine, threonine, lysine, arginine, histidine, aspartic acid, glutamic acid, and cysteic acid;

n is an integer from 0 to 10;

Aₘ is selected from the group consisting of a covalent bond and methionine;

P_{Hc2} is a peptide with the general formula Aₘ(A_{H})ₙA_{c},

wherein A_{c}, if y is 1, is selected from the group consisting of a covalent bond, ornithine, cysteine, homocysteine, cysteic acid, and lysine; or, if y is 0, is a terminating group selected from the group consisting of alcohol moieties, amine moieties, ester moieties, ether moieties, carboxylic acid moieties, amide moieties, and sulfonic acid moieties;

each of A_{H} is, independently, a charged or uncharged hydrophilic amino acid selected from the group consisting of serine, threonine, lysine, arginine, histidine, aspartic acid, glutamic acid, and cysteic acid;

n is an integer from 0 to 10;

Aₘ, is selected from the group consisting of a covalent bond and methionine;

P_{S} is a peptide from 5 to 25 amino acids in length;

T is a terminating group selected from the group consisting of alcohol moieties, amine moieties, ester moieties, ether moieties, carboxylic acid moieties, amide moieties, sulfonic acid moieties, quencher moieties, and detectable moieties (preferred detectable moieties being a fluorescent moiety, a hapten moiety, a biotin moiety, a chromogenic substrate moiety, or a chemiluminescent substrate moiety, and most preferably a fluorescent moiety different from *F); and

y is 0 or 1.

In another aspect, the invention is also drawn to a method of making the substrates of the invention by reacting at least one synthetic peptide, optionally a library of synthetic peptides, with the general formula:

P_{Hc1}-P_{S}-P_{Hc2}-(R₃-L₂-P₄-T)_{y}

wherein P_{Dc1} is a peptide with the general formula A_{c} (A_{H})ₙAₘ,

wherein A_{c} is a coupling amino acid selected from the group consisting of cysteine, cysteic acid, and homocysteine;

each of A_{H} is, independently, a charged or uncharged hydrophilic amino acid selected form the group consisting of serine, threonine, lysine, arginine, histidine, aspartic acid, glutamic acid, and cysteic acid;

n is an integer from 0 to 10;

Aₘ is a covalent bond or methionine;

P_{Hc2} is a peptide with the general formula Aₘ(A_{H})ₙA_{c},

wherein A_{c}, if y is 1, is selected from the group consisting of a covalent bond, ornithine, cysteine, homocysteine, cysteic acid, and lysine; or, if y is 0, is a terminating group selected from the group consisting of alcohol moieties, amine moieties, ester moieties, ether moieties, carboxylic acid moieties, amide moieties, and sulfonic acid moieties;

each of A_{H} is, independently, a charged or uncharged hydrophilic amino acid selected from the group consisting of serine, threonine, lysine, arginine, histidine, aspartic acid, glutamic acid, and cysteic acid;

n is an integer from 0 to 10;

Aₘ, is selected from the group consisting of a covalent bond and methionine;

P_{S} is a peptide from 5 to 25 amino acids in length;

R₃ and R₄ are each, independently: a covalent bond or a covalent linkage consisting of a branched or unbranched, substituted or unsubstituted, saturated or unsaturated chain of 1-10 carbon atoms; 0-3 heteroatoms selected from the group consisting of oxygen, nitrogen, and sulfur; and further consisting of at least one linkage chosen from the group consisting of ether, ester, hydrazone, amide, thioether, thioester, thiourea, disulfide and sulfonamide linkages;

L₂ is a branched or unbranched hydrophilic uncharged polymer selected from the group consisting of polyethylene glycol (PEG) and polysaccharides having a molecular weight of about 80 to about 4000 Daltons, more preferably from about 100 to about 2000 Daltons, more preferably from about 500 to about 1500 Daltons;

T is a terminating group selected from the group consisting of alcohol moieties, amine moieties, ester moieties, ether moieties, carboxylic acid moieties, amide moieties, sulfonic acid moieties, quencher moieties, and detectable moieties (preferred detectable moieties being a fluorescent moiety, a hapten moiety, a biotin moiety, a chromogenic substrate moiety, or a chemiluminescent substrate moiety, and most preferably a fluorescent moiety different from *F); and

y is 0 or 1,

with at least one labeled hydrophilic polymer with the general formula;

*F-R₁-L₁-X

wherein *F is a detectable moiety with a molecular weight of less than 5 kD, preferably a fluorescent, chemiluminescent, or chromogenic detectable moiety;

R₁ is a covalent bond or a covalent linkage consisting of a branched or unbranched, substituted or unsubstituted, saturated or unsaturated chain of 1 -10 carbon atoms; 0-3 heteroatoms selected from the group consisting of oxygen, nitrogen, and sulfur; and further consisting of at least one linkage chosen from the group consisting of ether, ester, hydrazone, amide, thioether, thioester, thiourea, disulfide and sulfonamide linkages;

L₁ is a branched or unbranched hydrophilic uncharged polymer selected from the group consisting of polyethylene glycol (PEG) and polysaccharides having a molecular weight of about 80 to about 4000 Daltons, more preferably from about 100 to about 2000 Daltons, more preferably from about 500 to about 1500 Daltons; and

X is a reactive moiety consisting of 0 to 10 carbon atoms; 0-6 heteroatoms selected from the group consisting of oxygen, nitrogen, and sulfur; and further consisting of at least one selectively reactive electrophilic group selected from the group consisting of: Br, Cl, I, n-hydroxyl succinimimidyl ester, and pyridyldisulfide.

In another aspect, the invention is drawn to a method of making the substrates of the invention by a modification of the conventional solid phase polypeptide synthesis methods. The method comprises adding a reagent with the general structure:

Pct-NH-R₅-L₁-R₆-COOH

wherein Pct is a protecting group, preferably fluorenylmethyloxycarbonyl (FMOC), butyloxycarbonyl (BOC), or another acid labile protecting group;

R₅ and R₆ are each, independently: a covalent bond or a branched or unbranched, substituted or unsubstituted, saturated or unsaturated chain of 1-10 carbon atoms and 0-3 heteroatoms selected from the group consisting of oxygen, nitrogen, and sulfur; and

L₁ is a branched or unbranched hydrophilic uncharged polymer selected from the group consisting of polyethylene glycol (PEG) and polysaccharides having a molecular weight of about 80 to about 4000 Daltons, more preferably from about 100 to about 2000 Daltons, more preferably from about 500 to about 1500 Daltons,

to the solid support reaction, either before and/or after synthesizing the peptide portion of the substrate, so as to add the hydrophilic polymer to the C-terminus and/or the N terminus of the peptide being synthesized on the solid support. The small detectable group (fluorophore, chromogenic moiety, or other detectable moiety) may be added to the structure on the solid support before and/or after the addition of the hydrophilic polymer reagent and synthesis of the peptide, or added to a linking group on end of the hydrophilic polymer-peptide structure after cleavage from the solid support.

In yet another aspect, the invention is drawn to methods of using the substrates of the invention in electrophoretically based enzymatic activity assays, preferably in protein-kinase, protein-phosphatase, or protease-activity assays, to determine the effect of a potential inhibitor or activator of the reaction on the kinase, phosphatase, or protease. Fluorescent detection is preferred in the assay methods of the invention.

In protein-kinase and protein-phosphatase assay embodiments, the method generally comprises:

(a) combining the molecule of interest, an enzyme selected from the group consisting of protein-kinases and protein-phosphatases, and one or more peptidic substrates of the invention, wherein a Ps comprising a recognition sequence for the protein kinase is within one or more of the peptidic substrates, under conditions suitable for the activity of the enzyme (e.g., buffers, temperature, ATP, cofactors, etc.);

(b) terminating the activity of the enzyme after a period of time;

(c) electrophoretically separating the phosphorylated peptidic substrate from the unphosphorylated peptidic substrate to produce a localized phosphorylated peptidic substrate fraction and unphosphorylated peptidic substrate fraction; and

(d) quantifying at least one of the separated fractions by detecting a detectable moiety on the peptidic substrate in the localized fraction, thereby determining the extent of conversion of the substrate by the enzyme during the period of time.

In order to compare the effects of the molecule of interest with the natural action of the enzyme, and additional step (e), comparing the extent of conversion of the substrate by the enzyme in step (d) with the extent of conversion by the enzyme when the enzyme is combined with the peptidic substrate under conditions suitable for the action of the enzyme for a substantially identical period of time in the absence of the molecule of interest, may be performed. Alternatively, the effects of the molecule of interest may be compared with the effects of known stimulators or inhibitors of the enzyme.

Protein-phosphatase assays differ from protein-kinase assays in that the peptidic substrates are initially phosphorylated when added to the assay mixture for protein-phosphatase assays, and are subsequently dephosphorylated by the protein phosphatase. In protein-kinase assays, the peptidic substrates are added to the assay mixture as unphosphorylated peptidic substrates, and are then phosphorylated by the enzymatic reaction. In preferred embodiments of the protein-kinase and protein-phosphatase assay methods of the invention, the peptidic substrate carries a positive charge or no charge when unphosphorylated, and carries a negative charge when phosphorylated in order to facilitate electrophoretic separation of the products and reactants.

In protease-activity assay embodiments, the method is analogous to the above described kinase embodiments except that the peptidic substrate is cleaved rather than phosphorylated. Thus, in preferred embodiments of the invention, the peptide substrate carries a different charge before cleavage than its charge after cleavage. In particularly preferred embodiments, P_{Hc1} has a charge opposite that of P_{Hc2}, so as to create two cleavage products with charges different from that of the intact peptidic substrate. In addition, it is also preferred that the peptidic protease substrates of the invention for use in these methods have two different detectable moieties (*F and T in the above general structure) at either end of the molecule so that the cleavage event may be more easily studied. For instance, the peptidic substrates may have two moieties which fluoresce at different wavelengths, or a fluorescent moiety and a quencher moiety.

In another aspect, the present invention is drawn to peptidic substrate libraries for screening to discover optimal substrates for any protein-kinase, protease, or other peptide-ligand enzyme. The libraries of the invention consist of a set of members having the general structure:

*F-R₁-L₁-R₂-P_{Hc1}-P_{S}-P_{Hc2}-(R₃-L₂-R₄-T)_{y}

wherein *F is a detectable moiety with a molecular weight of less than 5 kD, preferably a fluorescent moiety, a hapten moiety, a chromogenic moiety, or a chemiluminescent moiety, and most preferably a fluorescent moiety;

R₁, R₂, R₃, and R₄ are each, independently: a covalent bond or a covalent linkage consisting of a branched or unbranched, substituted or unsubstituted, saturated or unsaturated chain of 1-10 carbon atoms; 0-3 heteroatoms selected from the group consisting of oxygen, nitrogen, and sulfur; and further consisting of at least one linkage chosen from the group consisting of ether, ester, hydrazone, amide, thioether, thioester, thiourea, disulfide and sulfonamide linkages;

L₁ and L₂ are each, independently: a branched or unbranched hydrophilic uncharged polymer selected from the group consisting of polyethylene glycol (PEG) and polysaccharides having a molecular weight of about 80 to about 4000 Daltons, more preferably from about 100 to about 2000 Daltons, more preferably from about 500 to about 1500 Daltons;

P_{Hc1} is peptide with the general formula A_{c}(A_{H})ₙAₘ,

wherein A_{c} is selected from the group consisting of a covalent bond, ornithine, cysteine, homocysteine, cysteic acid, and lysine;

each of A_{H} is, independently, a charged or uncharged hydrophilic amino acid selected form the group consisting of serine, threonine, lysine, arginine, histidine, aspartic acid, glutamic acid, and cysteic acid;

n is an integer from 0 to 10;

Aₘ is selected from the group consisting of a covalent bond and methionine;

P_{Hc2} is a peptide with the general formula Aₘ(A_{H})ₙA_{c},

wherein A_{c}, if y is 1, is selected from the group consisting of a covalent bond, omithine, cysteine, homocysteine, cysteic acid, and lysine; or, if y is 0, is a terminating group selected from the group consisting of alcohol moieties, amine moieties, ester moieties, ether moieties, carboxylic acid moieties, amide moieties, and sulfonic acid moieties;

each of A_{H} is, independently, a charged or uncharged hydrophilic amino acid selected from the group consisting of serine, threonine, lysine, arginine, histidine, aspartic acid, glutamic acid, and cysteic acid;

n is an integer from 0 to 10;

Aₘ, is selected from the group consisting of a covalent bond and methionine;

P_{S} is a peptide from 5 to 25 amino acids in length;

T is a terminating group selected from the group consisting of alcohol moieties, amine moieties, ester moieties, ether moieties, carboxylic acid moieties, amide moieties, sulfonic acid moieties, quencher moieties, and detectable moieties (preferred detectable moieties being a fluorescent moiety, a hapten moiety, a biotin moiety, a chromogenic substrate moiety, or a chemiluminescent substrate moiety, and most preferably a fluorescent moiety different from *F); and

y is 0 or 1.

In preferred embodiments for use in selecting substrates for protein kinase assays, the substrate peptide P_{S} consists of a partially random amino acid sequence, in which a central amino acid is serine, threonine, or tyrosine. In especially preferred embodiments of these libraries, P_{S} consists of 5 to 10 amino acids, more preferably 6-8 amino acids, and most preferably 7 amino acids. In preferred embodiments for use in selecting substrates for protein phosphatases, the substrate peptide P_{S} consists of a partially random amino acid sequence, in which a central amino acid is phosphorylated serine, phosphorylated threonine, or phosphorylated tyrosine. Phosphorylated amino acid residues may be synthesized within the peptide using conventional solid-phase synthesis techniques. In especially preferred embodiments of these libraries, P_{S} consists of 5 to 10 amino acids, more preferably 6-8 amino acids, and most preferably 7 amino acids.

In another aspect, the invention is also drawn to methods of selecting peptides for use in enzymatic activity assays from the libraries of the invention, particularly for use in protein kinase assays. The selection method of the invention generally comprises the steps of:

(a) separating the members of the library which are soluble under suitable reaction conditions for the protein-modifying enzyme from those which are not soluble under suitable reaction conditions for the protein-modifying enzyme;

(b) combining the soluble members of the library obtained in (a) with the protein-modifying enzyme under suitable reaction conditions for the protein-modifying enzyme, thereby modifying some members of the library;

(c) separating the modified members of the library produced in (b) from the unmodified members of the library;

(d) determining the sequence of P_{S} for the modified members of the library.

Preferred methods for separating the modified or phosphorylated members of the library from the unphosphorylated members of the library include metal chelation chromatography, chromatofocusing, and electrophoretic separation. Because of their ability to distinguish between peptides with charge characteristics suitable for use in the electrophoretic assays of the invention, the separation methods of chromatofocusing and electrophoretic separation are particularly preferred for use in the screening methods of the invention.

### BRIEF DESCRIPTION OF THE DRAWINGS

FIG. 1: Scan of an agarose slab gel containing fluorescent substrates treated with PKA and ATP. The gel is a horizontal slab of 0.8% agarose in 50 mM TRIS HCl, pH 8.0. The polarity of the electric field is indicated by the plus and minus signs. The bands were visualized by irradiation with ultraviolet light and measurement of fluorescence. Lane 1: Lissamine-labeled Kemptide, −PKA; Lane 2: Lissamine-labeled Kemptide, +PKA; Lane 3: Lissamine-labeled, synthetically phosphorylated Kemptide; Lane 4: Texas Red-labeled Kemptide, isomer 1, −PKA; Lane 5: Texas Red-labeled Kemptide, isomer 1, +PKA; Lane 6: Texas Red-labeled Kemptide, isomer 2, −PKA; Lane 7: Texas Red-labeled Kemptide, isomer 2, +PKA.

FIG. 2: Scan of an agarose slab gel containing fluorescent substrates treated with PKA and ATP. The gel is a horizontal slab of 0.8% agarose in 50 mM TRIS HCl, pH 8.0. The polarity of the electric field is indicated by the plus and minus signs. The bands were visualized by irradiation with ultraviolet light and measurement of fluorescence. Lanes 1 & 3: (BODIPY-PEG)-labeled Kemptide, −PKA; Lane 2: Lissamine-labeled Kemptide, −PKA; Lane 4: Blank; Lanes 5&7: (BODIPY-PEG)-labeled Kemptide, +PKA; Lane 6: Lissamine-labeled Kemptide, +PKA.

FIG. 3: Scan of an agarose slab gel containing fluorescent substrates treated with SRC kinase and ATP. The gel is a horizontal slab of 0.8% agarose in 50 mM TRIS HCl, pH 8.0. The polarity of the electric field is indicated by the plus and minus signs. The bands were visualized by irradiation with ultraviolet light and measurement of fluorescence. Lane 1: Texas Red-labeled, synthetically serine-phosphorylated (pS) Kemptide; Lane 2: Texas Red-labeled Kemptide; Lane 3: Blank; Lane 4: C-[S-(TXR-Jeff)]EEEFIYGAFKKKK [SEQ. ID NO. 1], +SRC kinase; Lane 5: Blank; Lane 6: C-[S-(TXR-Jeff)]EEEFI(pY)GAFKKKK [SEQ. ID NO. 2], synthetically phosphorylated; Lane 7: Blank; Lane 8: C-[S-(TXR-Jeff)]EEEFIYGAFKKKK [SEQ. ID NO. 1], -SRC kinase.

FIG. 4: Scan of an agarose slab gel containing fluorescent substrates treated with SRC kinase and ATP. The gel is a horizontal slab of 0.8% agarose in 50 mM TRIS HCl, pH 8.0. The polarity of the electric field is indicated by the plus and minus signs. The bands were visualized by irradiation with ultraviolet light and measurement of fluorescence. Lane 1: C-[S-(TXR-Jeff)]EEEFIYGAFKKKK [SEQ. ID NO. 1], −SRC kinase; Lane 2: C-[S-(TXR-Jeff)]EEEFIYGAFKKKK [SEQ. ID NO. 1], +SRC kinase; Lane 3: C-[S-(TXR-Jeff)]EEEFI(pY)GAFKKKK [SEQ. ID NO. 2], synthetically phosphorylated; Lane 4: Ac-C-[S-(BTR-Jeff)]EEFIYGAFKKKK [SEQ. ID NO. 3], −SRC kinase; Lane 5: Ac-C-[S-(BTR-Jeff)]EEFIYGAFKKKK [SEQ. ID NO. 3], +SRC kinase; Lane 6: Ac-C-[S-(BTR-Jeff)]EEFIYGAFRRRR [SEQ. ID NO. 4], −SRC kinase; Lane 7: Ac-C-[S-(BTR-Jeff)]EEFIYGAFRRRR [SEQ. ID NO. 4], +SRC kinase; Lane 8: Blank.

FIG. 5: Scan of an agarose slab gel containing fluorescent substrates treated with SRC kinase and ATP. The gel is a horizontal slab of 0.8% agarose in 50 mM TRIS HCl, pH 8.0. The polarity of the electric field is indicated by the plus and minus signs. The bands were visualized by irradiation with ultraviolet light and measurement of fluorescence. Lane 1: Texas Red-labeled Kemptide; Lane 2: Texas Red-labeled, synthetically serine-phosphorylated (pS) Kemptide; Lane 3: C-[S-(TXR-Jeff)]EEEFIYGAFKKKK [SEQ. ID NO. 1], −SRC kinase; Lane 4: C-[S-(TXR-Jeff)]EEEFIYGAFKKKKv, +SRC kinase; Lane 5: C-[S-(TXR-Jeff)]EEEFI(pY)GAFKKKK [SEQ. ID NO. 2], synthetically phosphorylated; Lane 6: Ac-C-[S-(TXR-Jeff)]EEFIYGAFKKKK [SEQ. ID NO. 5], −SRC kinase; Lane 7: Ac-C-[S-(TXR-Jeff)]EEFIYGAFKKKK [SEQ. ID NO. 5], +SRC kinase; Lane 8: Blank.

FIG. 6: Scan of an agarose slab gel containing fluorescent substrates treated with SRC kinase and ATP. The gel is a horizontal slab of 0.8% agarose in 50 mM TRIS HCl, pH 8.0. The polarity of the electric field is indicated by the plus and minus signs. The bands were visualized by irradiation with ultraviolet light and measurement of fluorescence. Lane 1: Blank; Lane 2: C-[S-(TXR)]EEEFIYGAFKKKK [SEQ. ID NO. 1], -SRC kinase; Lane 3: C-[S-(TXR)]EEEFIYGAFKKKK [SEQ. ID NO. 1], +SRC kinase; Lanes 4&7: C-[S-(TXR-Jeff)]EEEFI(pY)GAFKKKK [SEQ. ID NO. 2], synthetically phosphorylated; Lane 5: C-[S-(TXR-Jeff)]EEEFIYGAFKKKK [SEQ. ID NO. 1], −SRC kinase; Lane 6: C-[S-(TXR-Jeff)]EEEFIYGAFKKKK [SEQ. ID NO. 1], +SRC kinase; Lane 8: Blank.

### DEFINITIONS

As used herein, the term “substrate” or “peptidic substrate” means a detectable, solubilized peptidic substrate of the invention with the general structure *F-R₁-L₁-R₂-P_{Hc1}-P_{S}-P_{Hc2}-(R₃-L₂-R₄-T)_{y}, unless otherwise indicated by the context in which it is used. For consistency, *F, R₁, R₂, R₃ and R₄, R₅ and R₆, L₁ and L₂, P_{Hc1} and P_{Hc2}, T, Y, A_{c}, Aₘ, A_{H}, P_{S}, n, y, X, and Pct, as used in the general formulas in the specification, have the same meaning throughout as given to them in the summary of invention.

The term “solid phase peptide synthesis,” as used herein, means the chemical synthesis of a peptide by anchoring one end of the nascent peptide to a solid support (which may be porous, non-porous) in various formats (chromatography resins, dipsticks, wells, beads, membranes, etc.), and adding amino acid subunits (either individual amino acids or oligo-amino acids) by successive rounds of deprotection and amine-carboxylic acid condensation reactions. Included in this definition is the synthesis of peptides containing standard, modified (e.g., phosphorylated), rare (e.g., ornithine), and/or synthetic amino acid residues.

### DETAILED DESCRIPTION OF THE INVENTION

This invention concerns the design and preparation of water-soluble labeled peptide substrates for use in enzymatic activity assays. The peptidic substrates of the invention are readily separated from their phosphorylated counterparts by electrophoresis. These synthetic substrates are particularly useful in protein kinase, phosphatase and protease activity assays. Because of their relative stability (as compared to radioactive substrates), substrate sequence flexibility (because the substrate portion of the peptide is not relied upon to provide molecular hydrophilicity), and ease of use with standard fluorescent, calorimetric, or luminometric detection equipment, these substrates are also particularly suited to high throughput screening in combinatorial chemistry testing of potential kinase, phosphatase, or protease inhibitors or stimulators.

Peptidic Substrate Design

As described above, the modified synthetic peptide substrates (“substrates”) of the invention have the general formula F-R₁-L₁-R₂-P_{Hc1}-P_{S}-P_{Hc2}-(R₃-L₂-R₄-T)_{y}. The component moieties of the substrates (*F, P_{Hc1}-P_{S}-P_{Hc2}, L₁ and L₂, and T) are selected as described below in order to optimize the use of the substrate in non-radioactive enzyme activity assays, which are especially suitable for high-throughput screening techniques. The component moieties are then linked together utilizing conventional synthetic organic chemistry techniques, as described below, to form the peptidic substrate molecules.

The small (under 5000 Daltons) detectable moiety of the peptidic substrates may be any non-radioactive detectable moiety suitable for use in biological assays. Suitable moieties include fluorescent moieties, hapten moieties, chromogenic moieties (e.g., peroxidase substrate moieties), and chemiluminescent moieties (e.g., acridinium). Fluorescent, chemiluminescent, and colorimetric moieties are preferred because they may be directly detected, rather than relying on a primary binding event that is detected through a second detectable moiety (such as with haptens, biotin, or other affinity labels). Similarly, fluorescent moieties are most preferred for use as detectable moieties because they do not involve the addition of further reagents for detection. By employing a fluorescent label in the peptidic substrate, easy and sensitive detection may be accomplished using commercially available fluorescence detectors and plate readers, with the latter instrumentation allowing for the simultaneous measurements of samples in 96-well, 384-well, and 1536-well microtiter plates.

Fluorescent moieties for use in the present invention include active-ester or other reactive derivatives of BODIPY_{630/650} X-SE, Texas Red X-SE, or BODIPY TRX-SE, Cy-dyes, fluorescein, rhodamine, phycoerythrin, and coumarin. Because of the increased solubility of the peptidic substrates, sparingly soluble fluorophores that are not normally used as peptide labeles may be used in the substrates of the invention. The fluorophore should be chosen with consideration of its charge characteristics at the reaction or separation pH of the assay in which it will be used, as these may affect the final pI of the fluorescent peptidic substrate. For example, Texas Red contains ionizable groups that fall well outside of the pH range of typical electrophoresis, and thus does not have any impact on the pI of the substrate. These groups also help to solubilize the dyes. In addition, excitation and emission properties should be chosen so as to minimize interference from intrinsic fluorescence of the materials that comprise the assay device to be used. Dyes such as Lissamine and Texas Red are desirable because their excitation wavelengths are high so as to minimize the interference caused by the intrinsic fluorescence of assay devices.

A key feature of this invention is the employment of an uncharged hydrophilic polymer group to link the relatively hydrophobic label molecule to the substrate peptide, and thus increase the solubility of the entire substrate molecule without regard to the solubility of the detectable moiety or the peptide. Several hydrophilic polymers which are commonly used for protein derivatization are commercially available for use in the present invention, including polyethylene glycol and polysaccharides. Various lengths of hydrophilic polymers may be employed, ranging in size from about 80 Daltons (two ethylene glycol units) to about 4000 Daltons, more preferably from about 100 to about 2000 Daltons, more preferably from about 500 to about 1500 Daltons, and most preferably from about 800 to about 1000 Daltons. Polyethylene glycol polymers ranging in size from about 230 to about 2000 Daltons are particularly preferred for use in the invention. For example, polyethylene glycol (PEG) as obtained in the forms of the α,ω-diamino derivative Jeffamine ED-900 (used in Schemes 1 & 2), an α,107 -PEG amino acid (produced in Scheme 3), or another bifunctional oligo-ethlyene glycol unit, are suitable for use as linkers. The Jeffamine series of diamino PEG's ranges from 230 to 2000 molecular weight, and thus are useful for optimizing the length of the PEG for a particular peptidic substrate.

Use of larger PEG or polysaccharide linkers may be disfavored when the peptidic substrate will be used in an assay in which a molecular sieving material (e.g. agarose, polyacrylamide, or other hydrogels) is used for electrophoretically separating the reacted and unreacted substrates. Similarly, the effect of the hydrophilic polymer size on the charge/mass ratio (and thus on overall electrophoretic mobility) should be considered when choosing the hydrophilic polymer. It is preferred that the hydrophilic polymer moiety be used as a hydrophilic spacer to link a fluorophore to a synthetic peptide that can serve as a substrate for a protein kinase. In addition, the hydrophilic polymer moiety may be incorporated on the other side of the peptide sequence, so long as the ability of the substrate to be phosphorylated is not impaired. In this case, the additional hydrophilic polymer moiety may simply be flanked by a terminating group, or may also be linked to a quencher moiety or another detectable moiety.

In addition to the hydrophilic polymer linker, the structure of the peptidic portion of the molecule is important for the substrates of the invention. When the peptidic substrate is to be used in protein-kinase or protein phosphatase assays, the P_{S} amino-acid sequence of the peptide is designed to contain the phosphate-group acceptor e.g., serine, threonine, or tyrosine, that is flanked by the residues necessary for the recognition of the substrate by the kinase. Similarly, when the substrate is to be used in a protease reaction, P_{S} is designed to contain the protease recognition and cleavage site.

The amino-acid composition is further optimized so that the unphosphorylated fluorescent peptidic substrate (or cleaved substrate) contains a different charge than the phosphorylated substrate (or uncleaved substrate.) Preferably, the unphosphorylated substrate carries a net positive one charge. Upon phosphorylation by a protein kinase in the presence of ATP the net charge of the peptidic substrate becomes negative one. Similarly, the substrate may be designed to contain a neutral charge when unphosphorylated and a negative two charge when phosphorylated, or to have a positive two charge when unphosphorylated, and a neutral charge when phosphorylated. This charge difference allows for the facile separation of the substrate and product in an electric field or by ion-exchange chromatography.

When the substrate is to be used in a protease assay, it us useful to separate the charge of the molecule on either side of the PS peptide, so that P_{Hc1} has a charge opposite that of P_{Hc2}. If these charges are balanced, then the uncleaved substrate is neutral, one side of the cleaved substrate migrates towards the anode, and the other side migrates towards the cathode. If y=1, and T is a detectable moiety, both sides of the cleaved substrate may then be separated and detected for analysis. This is especially useful when screening libraries of peptides to obtain the protease recognition site. Alternatively, the substrate may be designed so that the charges of P_{Hc1} and P_{Hc2} are opposite and unbalanced, with the lesser charge in P_{Hc1}. In this design, the uncleaved substrate has an opposite charge of a cleaved portion of the substrate which contains the *F label, thus allowing the cleaved substrate signal to be rapidly separated from the uncleaved substrate. A less preferred design is that where the greater charge of an unbalanced P_{Hc1}/P_{Hc2} pair is in P_{Hc1}. In this design, the cleaved peptide substrate with the detectable moiety *F may be differentiated from the uncleaved substrate by the rate of migration during electrophoresis.

In order to convey charge on the peptide portions of the substrates, appropriately charged hydrophilic amino acids are added to P_{Hc1} and P_{Hc2}, in the (A_{H})ₙ portions of these subsequences. Amino acids that have acid dissociation constants within the range of 5 to 9 are undesirable because they move the pI of the peptides closer to the pH range of the electrophoretic separation and thereby reduce the rate of migration of the peptides. These amino acids include histidine and cysteine, although histidine may be used if necessary. More suitable positively charged amino acids for use as A_{H} include lysine, arginine. Most preferred for use is lysine. Suitable positively charged amino acids for use as A_{H} include aspartic acid, glutamic acid, and cysteic acid. Most preferred for use is glutamic acid.

In addition, the polar uncharged amino acids serine or threonine may be added to further solubilize the peptidic portion of the substrate. Obviously, as these residues are modified by some protein-kinases, care should be taken not to include these residues in the P_{Hc} sequences if the substrate is to be used in a protein-kinase assay where serine or threonine would be modified. In general, it is preferred that strings of the same amino acid be utilized as the (A_{H})ₙ portions of the P_{Hc} sequences (e.g., tri-glutamic acid or tri-lysine). Other amino acids such as cysteic acid for inclusion in at selected points in the sequence and 2-aminoethane sulfonic acid for inclusion at the carboxy terminus (in cases where y=0, and R₂ is attached to the N-terminus) could be used to increase water-solubility. Sulfonic acid modifications are often used to increase the water-solubility of hydrophobic molecules. The acid-dissociation constants of sulfonic acids are quite low and would not adversely affect the pI of the fluorescent peptidic substrate.

In addition to (A_{H})ₙ portions of the P_{Hc} sequences, coupling (A_{c}) and labile (Aₘ) amino acids may be added to the peptide sequence in order to provide reactive moieties for linkage chemistries or cleavage chemistries. Particularly, cysteine, homocysteine, ornithine and lysine are useful as A_{c}. Cysteine and homocysteine provide reactive sulfhydryl groups that are especially useful in nucleophilic substitution reactions, as described in the synthesis discussion below. Ornithine and lysine are also useful, as they provide reactive amine groups for attachment chemistries. In addition, the inclusion of a methionine residue, Aₘ, is preferred in library embodiments to provide a chemical cleavage site for uncovering the sequence of the reactive members of the library. As described below, methionine sequences may be chemically cleaved using cyanogen bromide, allowing the remainder of the reactive substrate sequence to be determined by traditional Edman degradation or by carboxy-terminal degradation of the peptide.

The principles of designing an appropriately charged peptidic portion of the molecule are illustrated by the following, with reference to the examples. The design of the exemplary engineered amino-acid sequence began with a screen of a library of resin-bound peptides that determined the sequence FIYGAFK [SEQ. ID NO. 6] to be an active substrate of SRC Kinase. This screen was done by investigators at Selectide, who provided the applicants with the sequence information. See, .e.g., U.S. Pat. No. 6,090,912. Additional residues were added to the amino terminus to provide a thiol for chemoselective coupling of a fluorophore, yielding the sequence CAAFIYGAFK [SEQ. ID NO. 7]. Insolubility of this peptide in its underivatized form required the addition of charged amino acids so the sequence CEEEFIYGAFKKKK [SEQ. ID NO. 8] was prepared. The pH at which electrophoretic separation step for the SRC kinase assay is in the range of 7-8. Thus, it is necessary to design a fluorescent peptidic substrate that has a pI greater than 9, so that upon phosphorylation by SRC kinase the pI becomes less than 6. This ensures that the net charge of the unreacted substrate is positive and net charge of the phosphorylated substrate is negative. With this in mind three lysine residues were added to the carboxy terminus, and three counterbalancing glutamic acid residues were added to amino terminus. Conjugation of a net neutral dye to the thiol of the cysteine yields a fluorescent peptide that has an estimated pI of 9.

To further increase the water-solubility of the fluorescent peptidic substrate an uncharged hydrophilic spacer was inserted between the fluorophore and the amino terminus. Jeffamine ED-900 was chosen as the PEG spacer because it can easily be functionalized with a fluorophore and an electrophile so that it can be conjugated to a synthetic peptide. A conjugate of Texas Red and the peptide CEEEFIYGAFKKKK [SEQ. ID NO. 8] linked with a Jeffamine ED-900 spacer was synthesized. The synthetic schemes of the Texas Red-Jeffamine₉₀₀-bromoacetamide and the Texas Red-Jeffaminego₉₀₀-peptide conjugate appear in Scheme 1 and Scheme 2 of Example 2, respectively. This fluorescent peptide was assayed and found to be a good substrate for SRC Kinase (Kₘ=8.5 μM).

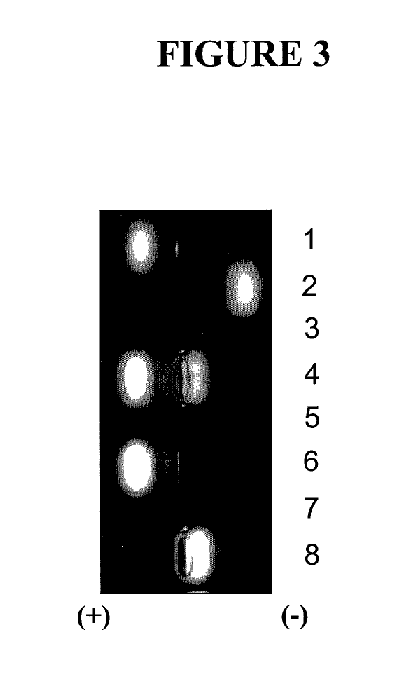

The electrophoretic mobility of this fluorescent peptide was acceptable, as shown in FIG. 3, but it was determined that a pI of 13 would increase the mobility of the peptidic substrate in the electrophoretic separation media. A glutamic acid was left out to increase the pI and mobility of the peptide. The sequence Ac-CEEFIYGAFKKKK [SEQ. ID NO. 9] was prepared where the amino terminus was blocked with an acetamide. The estimated pI is raised to 10, and the Jeffamine modified substrate has good reactivity and mobility, as demonstrated in Example 6. Replacement of the lysines with arginines further raised the pI to approximately 11.5. However, as demonstrated in Example 7, this alteration did not result in a sufficiently reactive substrate for SRC kinase.

Peptidic Substrate Preparation

In order to make the substrates of the invention, conventional selective linkage chemistries may be utilized, or solid-phase synthesis methods may be used. For the production of larger quantities of substrates for use in high-throughput screening assays, it is usually desirable to utilize a solution reaction scheme, such as the nucleophilic substitution reaction described below, utilizing a solid-phase synthesized polypeptide as a reactant. When libraries of peptide substrates are to be produced, solid phase synthesis of the entire molecule may be favored, in which a protected hydrophilic polymer α,107 amine carboxylic acid reagent is used.

The water-soluble fluorescent peptide substrate may be assembled from a fuilly deprotected synthetic peptide P_{Hc1}-P_{S}-P_{Hc2}-(R₃-L₂-R₄-T)_{y}, wherein P_{Hc1} is a peptide comprising a coupling amino acid A_{c}, which is either cysteine or homocysteine, and a detectable moiety-hydrophilic polymer reagent *F-R₁-L₁-X, where X is an electrophilic group that is capable of reacting regio-selectively with the —SH nucleophile present in the synthetic peptide. In the following examples the electrophile is a bromoacetamide, and the nucleophile in the peptide is a thiol from the amino acid cysteine. However, other electrophiles such as iodoalkyl, cloroalkyl, N-hydroxyl succinimidyl ester or pyridyldisulfide groups, may be used as well. Similarly, other selectable nucleophilic groups on the coupling amino acid could be utilized, such as the primary amines of lysine or ornithine. The nucleophilic substitution reaction is carried out under standard conditions to produce the peptidic substrates. In substrates produced by this method, The *F-R₁-L₁- portion of the substrate is linked to the P_{Hc1}-P_{S}-P_{Hc2}-(R₃-L₂-R₄-T)_{y} portion of the substrate through an amide (if the nucleophile is an amine) or thioether (if the nucleophile is a sulfhydryl) linkage R₂ to the side chain of A_{c} of P_{Hc1}.

Alternatively, solid phase synthesis may be utilized to produce the entire peptidic substrate molecule. In these methods, standard solid phase peptide chemistries are used to add a hydrophilic polymer linker between the peptidic portion of the molecule and the detectable moiety *F, and also between the peptidic portion and T, if present. The hydrophilic polymer linker reagent utilized in these methods has the general formula:

Pct-NH-R₅-L₁-R₆-COOH

as defined in the summary of invention. Preferred protective groups Pct include FMOC and BOC, both of which are compatible with conventional peptide synthesis chemistries. This reagent is utilized in a manner similar to protected amino acids in solid phase synthesis. First, a solid support with reactive anchoring groups is provided. The first reagent is then anchored to the solid support in an anchoring reaction which produces a bond which is cleavable under peptide-bond-stable conditions.

If y=1, this first reagent may be T or a precursor to T (or *F or a precursor to *F, if the *F portion of the molecule is on the carboxyl side of the peptide portion of the molecule.) After the initial anchoring reaction, any protecting groups on T are removed, and the carboxylic acid group on the hydrophilic polymer linkage reagent is reacted with a reactive group on T. The protecting group Pct on the hydrophilic linker is then removed, and the first amino acid of the sequence P_{Hc2} is added as a primary amine protected reagent. The carboxylic acid of this first amino acid is then allowed to react with the deprotected amine of the hydrophilic polymer linker, thus beginning the synthesis of the peptidic portion of the substrate. the remainder of the peptidic portion of the molecule is synthesized according to the standard method. After the last amino acid of P_{Hc1} is added to the peptide, and is deprotected, the hydrophilic polymer linking reagent is again added, the carboxylic acid group forming an amide bond with the terminal amine of the peptide. The Pct group of the linking reagent is then removed, and the *F moiety added by an appropriate coupling reaction to the deprotected amine of the linking reagent. The reaction used to attach *F should take into account the prevention of side-reactions with any of the amino acid residues of the peptidic portion of the molecule. After the complete molecule is assembled, any protecting groups on the amino acid residue side chains are removed and the bond to the solid support is cleaved.

If y=0, the synthesis proceeds in the same manner as above, except that either 1) if the *F portion of the substrate is to be on the amino side of the peptidic portion of the substrate, the first amino acid of P_{Hc2} is reacted with the anchoring group on the solid support, and only the second hydrophilic polymer linking reaction is carried out; or 2) if the *F portion of the substrate is to be on the carboxyl side of the peptidic portion of the substrate, *F is reacted with the anchoring group of the solid support instead of T, and the synthesis of the molecule terminates with the addition of the last residue of P_{Hc2} (synthesizing the peptide in the reverse of the order set out above). It should be noted that the amino acid reagents may also be added as N-protected di-, tri-, or oligopeptides, which can speed assembly. This is particularly useful for adding whole P_{HC} sequences, or for adding pre-synthesized random or partially random P_{S} sequences for the generation of libraries.

In addition to the methods set forth above, other methods of producing the substrates of the invention will be readily apparent to those of ordinary skill in the art. Although some chemistries for linking the components of the substrate molecules together may be more convenient (such as the amide chemistry for solid phase synthesis), several equally feasible interchangeable chemistries have been developed to link detectable molecules (especially fluorophores,) hydrophilic polymers (especially PEG's,) and peptides. Thus, a daunting number of predictably reliable synthesis schemes can be created by simply combining compatibly derivatized components of the substrates of the invention.

Peptidic Substrate Library Design, and Use in Selecting Optimal Substrates

A particularly useful aspect of the peptidic substrates of the invention is that their solubility, electrophoretic mobility, and their ability to be detected are effectively independent of the particular amino acid sequence of the peptide substrate sequence, P_{S}. Thus, they may be used to design a degenerate library of water-soluble, peptidic, and detectable substrates that can be readily separated from their phosphorylated, dephosphorylated or cleaved counterparts by electrophoresis. Such libraries are useful for screening for substrates to use in protein-kinase, protein-phosphatase, and protease assays. In general, the library of fluorescent or otherwise detectably labeled) peptides is first fractionated by water solubility and then by isoelectric point (pI) using chromatofocusing. The fraction that elutes at a pI greater than 8 is treated with the enzyme of interest under conditions suitable for the action of the enzyme. The treated fraction is then submitted to either chromatofocusing, electrophoresis, or metal-chelate chromatography to isolate the phosphorylated, dephosphorylated, or cleaved peptides. The modified peptides then are treated with cyanogen bromide in order to unmask the amino terminus (if the amino terminus is blocked, and a methionine residue is included in the peptide) and finally submitted to Edman peptide sequencing. These screens have a significant advantage in that ready-to-assay substrates (complete with fluorophores, charge modifiers, and solubilizing groups) are identified, without the need for further modification. The identified substrate may then be manufactured en mass for high-throughput screening without the need for further research with derivatives.

Each member of the libraries of the invention has the general formula F-R₁-L₁-R₂-P_{Hc1}-P_{S}-P_{Hc2}-(R₃-L₂-R₄-T)_{y}, wherein P_{S} is the substrate peptide which is to be modified by the enzyme (protein-kinase, protein-phosphatase, or protease) in the library screen. The non-P_{S} portions of the molecule are identical or substantially identical, for all members of the library, allowing the members to be screened for sequence-specific interactions with the enzyme. The selection of the P_{S} portion of substrates in the library will depend on the enzyme of interest. If the target of the protease, kinase, or phosphatase is known, then Ps may comprise a portion of the target protein's amino acid sequence. This portion may be generated by synthesis of each peptide sequence (if sequence information is known,) or by chemical or enzymatic cleavage of the protein (e.g., heat and acid degradation, pepsin or papain digestion, etc.).

When the target of a protease is unknown, completely random peptide sequences, which contain equal distributions of all possible amino acids (with the possible exclusions, as detailed below) are useful. This allows all possible recognition sequences for the protease to be simultaneously explored in equal distribution. If limited information is known about the target of a protease (i.e., it is a membrane bound or a secreted protein), then a weighted random sequence may be used. In these sequences, different proportions of amino acids are used in the synthesis mixture, creating an unequal distribution of the occurrence of each amino acid in the members of the library. Thus, a library enriched for non-polar or polar amino acid containing sequences may be produced. A partially random amino acid sequence, in which a central amino acid chosen from a smaller pool of possible amino acids is flanked by randomly chosen amino acids, is useful for phosphatase and kinase assays. For these enzymes, the P_{S} amino-acid sequence of the members of the libraries is designed to contain a phosphate-group acceptor (e.g., serine, threonine, tyrosine, or phosphorylated derivatives thereof for phosphatase assays) that is flanked by one or two degenerate groups of amino acids.

The number of possible degenerate amino acids in the P_{S} sequence is limited by 1) the number of amino acids that can be incorporated by solid-phase peptide synthesis, and 2) by the ability to detect the reactive substrates in the library as a fraction of the total substrate population. For example, A mixture of nonapeptides with the central amino acid defined as one of the specific phosphate acceptors (serine, threonine, or tyrosine) with all possible naturally occurring amino acids at the degenerate positions contains 20⁸ (2.56×10¹⁰ ) unique sequences. If the incorporation of any amino acid at any position happens with equal probability, then in a 1 mmol pool there would be 2.35×10¹⁰ molecules or 39 femtomoles per unique sequence. Needless to say it would be difficult at best to identify any unique sequence from the 1-mmol preparation of degenerate nonapeptides. Reducing the number of possible amino acids from 20 to 10 reduces the number of unique sequences 256 times to 1.00×10⁸ and increases the amount of each unique sequence to 10 picomoles, a level near the limit of detection for amino-acid analysis and amino-terminal protein sequence analysis. Reducing the length of the peptides from nine to seven decreases the number of unique sequences to 20⁶ (64 million). In a 1 mmol pool there would be 9.41×10¹² copies (15.6 pmols) of each unique sequence. Reducing the possible amino acids from 20 to 10 increases the number of each unique sequence to 1 mol.

As illustrated from this example, it is preferable to limit both the size and the degeneracy of the members of the library in order to ensure that any substrate modified by the enzyme will be present in detectable and sequenceable amounts. Thus, it is preferred that P_{S} be between 5 and 25 amino acids long, and more preferably between 5 and 10 amino acids long, most preferably 7 or 8 amino acids long. In addition, it is preferred that a less than full complement of naturally occurring amino acids be utilized in the random or weighted random portions of the sequence. Residues for use at the degenerate positions in libraries for screening phosphatases or kinases preferably include the following: aspartic acid, asparagine, glutamic acid, glutamine, proline, glycine, alanine, valine, isoleucine, leucine, phenylalanine, lysine, and arginine. Serine, threonine, proline, methionine, tyrosine, tryptophan, and histidine were omitted for various reasons. Serine, threonine, and tyrosine may be omitted because they are phosphate acceptors. Cysteine and histidine may be omitted because their side chains have pKa values too close to the pH at which electrophoresis is performed, and because cysteine may be used in specific linkage chemistries elsewhere in the substrate molecule. Methionine is omitted because the cyanogen bromide cleavage, if used to reveal an amino terminus for sequencing, has to occur only at designated site. If the investigator insists on including methionine and needs to perform the cyanogen bromide cleavage, the isosteric replacement norleucine can be used in the place of methionine as norleucine does not undergo cyanogen bromide cleavage:

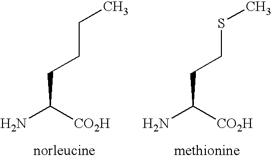

Other nonproteogenic amino acids may also be included in these peptidic constructions in order to optimize further the substrate activity. Tryptophan was omitted because it can be problematic in synthesis (it could be included if necessary). In a septapeptide with a central serine, threonine, or tyrosine, the number of unique sequences using the above list of degenerate amino acids would be 13⁶, which equals 4.83×10⁶: thus in a 1 mmol synthesis there would be 200 picomole of each unique sequence.

In a general method of screening the libraries of for suitable substrate molecules for a particular enzymatic assay:

1. The synthesized peptides are first fractionated based on water solubility.

2. The water-soluble fraction is then treated with the enzyme under conditions suitable for the action of the enzyme (i.e., ATP, appropriate buffer, appropriate temperature, etc.).

3. The reaction mixture is then subjected to metal-chelate chromatography, electrophoretic separation, chromatofocusing, or another fractionation step.

4. The separated modified substrate fraction is then be treated with CNBr to cleave a methionine in a peptide PHC sequence.

5. The CNBr-treated peptides are sequenced to determine the identity of the reactive substrates.

The first step in screening the substrate library, fractionating the library according to its solubility, is easily achieved by dissolving the peptide library in the reaction and separation buffers, allowing any insoluble members to precipitate, and filtering or decanting the solution. The substrate library is then reacted with the enzyme of interest (protein-kinase, protein-phosphatase, or protease) according to the proposed assay procedure. See the discussion of enzyme assay design, infra. If the first attempt to isolate a workable substrate does not succeed, lengthening of the reaction/incubation time may be appropriate. Similarly, several peptide libraries (with different P_{S} sequence structures or amino acid distributions, different variations in P_{HC} sequences, different hydrophilic polymer linkers, and different detectable moieties) may be screened in order to determine the best substrate for a particular enzyme assay.

After reaction with the enzyme, selective isolation of the modified substrates may be accomplished by several means. Metal-chelation chromatography was used by Singyang, et al, Current Biology, 4:973-82 (1994), to isolate phosphorylated peptides from a library of peptides that had been treated with a protein kinase in the presence of adenosine triphosphate (ATP). The stationary phase used in this separation technique consists of agarose beads that are derivatized with iminodiacetic acid. The column is charged with Fe⁺, and then the peptide mixture is applied. Alternatively, Ga⁺³ may be used as the charging cation. The phosphorylated peptides are retained on the column, chelated to the bound iron, while the unphosphorylated peptides pass through. After thorough washing the bound peptides are eluted from the column. If a protein-kinase is being assayed, a determination of the bound peptide sequences may yield a consensus sequence for the best substrate. Conversely, the initially eluted substrates would be sequenced for a protein-phosphatase assay.

Another chromatography method that may be used to isolate phosphorylated peptides, or cleaved and uncleaved substrates with a significant charge differential between P_{Hc1} and P_{Hc2}, is chromatofocusing. In this method an anion exchange resin is combined with a special buffer mixture that produces a linear pH gradient that allows molecules to be separated by their isoelectric points (pI). The pI of a peptidic substrate ideally should be greater than 9.0, and upon phosphorylation (or cleavage) the pI of the product peptide should be less than 7.0. Chromatofocusing may also be used to first fractionate the pool of potential peptidic substrates to obtain those whose pI's are greater than 9.0. This enriched fraction may then be treated with the enzyme and resubmitted to chromatofocusing. The phosphorylated peptides then elute at lower pH values and could then be identified by sequence analysis.

Polyarginine has been shown to selectively bind phosphorylated peptides in the presence of their unphosphorylated counterparts, and is a third possibility for the isolation of phosphorylated substrates. This binding reaction is used in a fluorescence-polarization assay for protein kinases developed by scientists at Caliper (Coffin, et al., “Detection of phosphopeptides by fluorescence polarization in the presence of cationic polyamino acids: application to kinase assays,” Analytical Biochemistry 278 (2):206-12 (2000).) Agarose beads that contain primary amino groups can be modified with 2-ethyl-2-thiopseudourea hydrobromide, yielding guanidino groups that may bind the phosphorylated peptide. Alternatively polyarginine may be conjugated to agarose to produce a stationary phase for phosphopeptide purification. Because the stability of the ionic interaction between the phosphorylated peptide and the stationary phase may be unpredictable, this approach is not preferred.

Lastly, electrophoresis can be used to separate phosphorylated peptide substrates from unphosphorylated substrates, or cleaved substrates from uncleaved substrates. This technique is especially preferred for use in the library screening methods of the invention as this method identifies those phosphorylated peptides that are electrophoretically mobile, a quality that the fluorescent peptidic substrate must possess in order to perform in several convenient enzyme assay formats. The enzyme-treated peptide library is simply subjected to an electric field, and those peptides that migrate towards the appropriate electrode are isolated for sequence analysis. For kinase reactions, the members of the library migrating towards the positive electrode, and thus containing additional negative charge (phosphate) would be collected. Conversely, the members of the library which travel towards the negative electrode would be collected for phosphatase reactions. If the substrates are designed to have oppositely charged P_{Hc1} and P_{Hc2} sequences, then those fractions of the library migrating more rapidly towards either or both electrodes will be collected for sequencing in protease reactions.

Once the fraction of modified substrates is collected, it is analyzed to determine the amino acid sequence of P_{S}. Analytical techniques exist that are capable of determining the sequences of picomoles of peptides. The most sensitive sequencer available from Applied Biosystems that employs the Edman Degradation can sequence samples as small as 200 femtomoles (1.20×10¹¹ molecules). High-resolution mass spectrometry can be used to obtain sequence information from samples as small as hundreds of picomoles. Although amino-acid analysis does not provide sequence information, the composition of the peptides may be used to further screen weighted random libraries.

In order to perform amino-terminal sequencing by the Edman Degradation the amino terminus must be unmodified. If the fluorophore-hydrophilic polymer linker portion of the substrate is coupled to the peptide portion of the substrate through the amino terminus, an alternative sequencing method must be used. Automated sequential carboxy-terminal sequencing can be performed with an ABI Procise C-Terminal Protein Sequencer. The limit of detection reported by the manufacturer is 500 picomoles, and the limit of readable sequence is 5-7 residues. Thus, it is theoretically possible to identify the at least partial sequences of phosphorylated peptides from a library by carboxy-terminal automated protein sequencing.

As only partial sequence information is given by carboxy-terminal sequencing, and the sequencer must first degrade the known P_{Hc} portion of the peptide sequence, it is preferable to be able to use the Edman degradation sequencing method. There is a way to unmask the amino terminus of a peptide by using methionine as an amino-terminal residue in all of the peptides in the library. After phosphorylation and subsequent isolation of the phosphorylated peptides, the amino termini can be unmasked by using cyanogen bromide to cleave the methionine residue. Under acidic conditions cyanogen bromide (CNBr) alkylates the thio-ether sulfur in the side chain of methionine. As a result, the beta carbon of methionine becomes electrophilic, and the carbonyl oxygen of the methionine residue attacks the beta carbon, resulting in cleavage of the peptide bond between methionine and the adjacent amino acid. This cleavage yields methyl thiocyanate, the fluorophore-linker, and the truncated peptide with a free amino terminus. The scheme of this reaction is shown below. The truncated peptide can then be sequenced using Edman sequencing conditions.

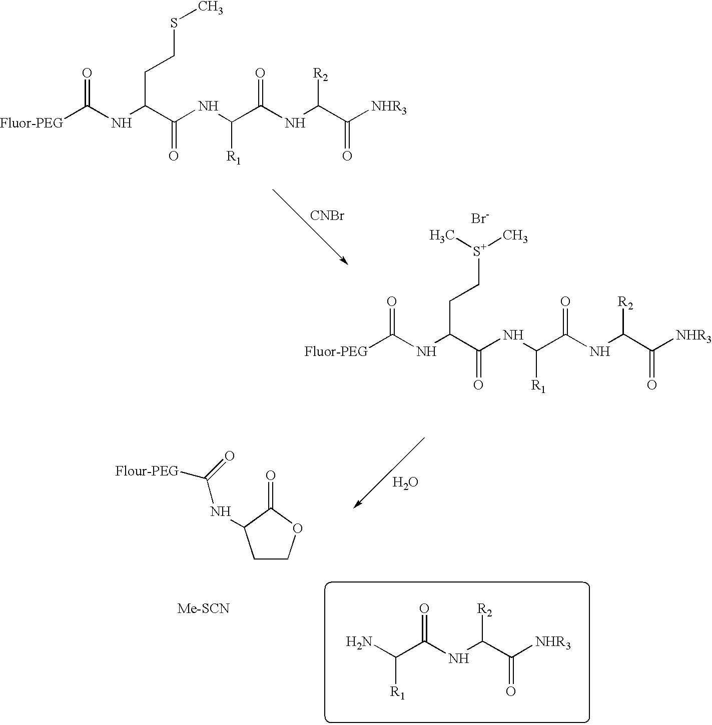

Once the P_{S} of the reactive substrate members of the library is determined, the sequence information may be used to directly produce a usable substrate for the enzymatic assay of interest. This first substrate may have sufficient desirable characteristics for use in the assay, or may be utilized as a starting point for further modifications to the non-PI, to fine tune its electrophoretic mobility, pI, or solubility.

Enzymatic Assay Methods Using the Peptidic Substrates of the Invention

The peptidic substrates of the invention are useful in a wide variety of enzymatic activity formats which employ charge discrimination to differentiate between the modified and unmodified substrate. In their simplest aspects, the modified and unmodified substrates may be separated using a molecular sieving medium, such as the agarose slab gels utilized in the Examples and shown in the Figures. More advanced devices for the electrophoretic separation of reactants and products are described in copending U.S. application Ser. No. 09/724,836, entitled “Microtiter Plate Format Device and Methods for Separating Differently Charged Molecules Using an Electric Field,” filed Nov. 28, 2000; Ser. No. 09/724,824, entitled “Microcapillary Arrays for High-Throughput screening and Separation of Differently Charged Molecules Using and Electric Field,” filed Nov. 28, 2000; and Ser. No. 09/724,909, entitled “Microstructure Apparatus and Method for Separating Differently Charged Molecules Using an Applied Electric Field,” filed Nov. 28, 2000, all of which are incorporated fully by reference herein.

These electrophoretically based enzymatic activity assays, in which the activity of a protein-kinase, protein-phosphatase, or protease is determined, are useful in the development of targeted pharmaceuticals which affect the activity of the enzyme. To determine the effect of a potential inhibitor or activator of the reaction on the kinase, phosphatase, or protease, the molecule of interest is added to a reaction mixture with the enzyme and a known substrate of the enzyme, and allowed to react under conditions suitable for the activity of the enzyme. The amount of the substrate which is converted or modified by the enzyme is then determined, and the extent of the effect of the substance of interest on the enzyme is determined.

In protein-kinase and protein-phosphatase assay embodiments, the method generally comprises:

(a) combining the molecule of interest, an enzyme selected from the group consisting of protein-kinases and protein-phosphatases, and one or more peptidic substrates of the invention, wherein a Ps comprising a recognition sequence for the protein kinase is within one or more of the peptidic substrates, under conditions suitable for the activity of the enzyme (e.g., buffers, temperature, ATP, cofactors, etc.);

(b) terminating the activity of the enzyme after a period of time;

(c) electrophoretically separating the phosphorylated peptidic substrate from the unphosphorylated peptidic substrate to produce a localized phosphorylated peptidic substrate fraction and unphosphorylated peptidic substrate fraction; and

(d) quantifying at least one of the separated fractions by detecting a detectable moiety on the peptidic substrate in the localized fraction, thereby determining the extent of conversion of the substrate by the enzyme during the period of time.

Usually, some of the working parameters for the enzyme will be known when the investigator is developing the assay. Suitable buffers and temperature will often be ascertainable from the enzyme function and its natural environment (e.g., except for thermophilic bacterial enzymes, 80° C. is usually not a suitable temperature, but 37° C. often is for human enzymes). The necessity of ATP, NADH, or other common co-substrates for the reaction will depend on the enzyme used in the assay. Kinases commonly require ATP as a phosphate source, for instance. Some enzymes may require metal ion such as Zn⁺², Mg⁺², Cu⁺², Fe⁺³ or coordinated complexes in order to function properly. If an investigator is capable of producing and isolating the functional enzyme, but does not have any information as to its functional conditions (e.g., a kinase “homolog” produced from a cloned gene picked from a general genome search), then suitable conditions for enzymatic activity may be ascertained by one of ordinary skill through screening combinations of condition variables which are suitable for homologous or similar proteins from the same organism.

The time chosen for the reaction period will also depend on the enzyme used in the assay, as well as on the type of effect studied. For some enzymes with robust activities, or for a study of potential stimulators of the enzyme, a short reaction time in the range of 15 minutes to 2 hours may be appropriate. For other assays, a medium reaction time in the range of 2 hours to 4 hours, a long reaction time in the range of 4 hours to 8 hours, or a very long reaction time in the range of 8 hours to 48 hours may be appropriate. The last category may be useful when screening for inhibitory compounds where a complete and irreversible inhibition is desired.

In order to compare the effects of the molecule of interest with the action of the enzyme in an unperturbed state, an additional step (e), comparing the extent of conversion of the substrate by the enzyme in step (d) with the extent of conversion by the enzyme when the enzyme is combined with the peptidic substrate under conditions suitable for the action of the enzyme for a substantially identical period of time in the absence of the molecule of interest, may be performed. This comparison may be in the form of a concurrently performed control assay, or may simply be a comparison of the current results for the molecule of interest with an average or median value obtained from past control assay data. Alternatively, the effects of the molecule of interest may be compared with the effects of known stimulators or inhibitors of the enzyme. These may also be in the form of concurrent positive control assays, or in the form of a numerical value obtained from past data. It should be noted that the use of concurrent assays is presently preferred, as slight variability in the conditions from test to test may cause drift in the absolute value of the data.

Protein-phosphatase assays differ from protein-kinase assays in that the peptidic substrates are initially phosphorylated when added to the assay mixture for protein-phosphatase assays, and are subsequently dephosphorylated by the protein phosphatase. In protein-kinase assays, the peptidic substrates are added to the assay mixture as unphosphorylated peptidic substrates, and are then phosphorylated by the enzymatic reaction. In preferred embodiments of the protein kinase and protein phosphatase assay methods of the invention, the peptidic substrate carries a positive charge or no charge when unphosphorylated, and carries a negative charge when phosphorylated in order to facilitate electrophoretic separation of the products and reactants.

In protease activity assay embodiments, the method is analogous to the above described kinase embodiments except that the peptidic substrate is cleaved rather than phosphorylated. Thus, in preferred embodiments of the invention, the peptide substrate carries a different charge before cleavage than its charge after cleavage. In particularly preferred embodiments, P_{Hc1} has a charge opposite that of P_{Hc2}, so as to create two cleavage products with charges different from that of the intact peptidic substrate. In addition, it is also preferred that the protease peptidic substrates of the invention for use in these methods have two different detectable moieties (*F and T in the above general structure) at either end of the molecule so that the cleavage event may be more easily studied. For instance, the peptidic substrates may have two moieties which fluoresce at different wavelengths, or a fluorescent moiety and a quencher moiety.

In all of the assays of the invention, the reaction mixture is separated by an electrophoretic step, allowing the modified and unmodified substrates to be differentiated by their position in the separation media or the device. This physical separation may be determined by detecting the detectable moiety on the substrate, either as an intensity in a flow path of the device over time (such as in capillary electrophoretic devices) or as the intensity of a signal in a particular place in the separation media or the device (such as the bands in the gels of the Figures.) Because of their ease of detection, easy of quantitation, and the wide variety of commercially available detection devices, fluorescent labels and detection are most preferred for use in the assay methods of the invention.

### EXAMPLES

The following examples are offered to further illustrate the various aspects of the present invention, and are not meant to limit the invention in any fashion. Based on these examples, and the preceding discussion of the embodiments and uses of the invention, several variations of the invention will become apparent to one of ordinary skill in the art. Such self-evident alterations are also considered to be within the scope of the present invention.

### Example 1

### Preparation of Fluorescent Peptide Derivatives for Comparison With The Peptidic Substrates of The Invention

Fluorescent derivatives of LRRASLG [SEQ. ID NO. 10] (Kemptide) were prepared with Texas Red-X, SE and Kemptide. Kemptide (2.4 mg) was dissolved in 300 μL of 100 mM sodium phosphate, pH 7.0. Texas Red-X, SE (5 mg) was dissolved in 300 μL of dry acetonitrile, and this solution was added to the peptide solution. The reaction proceeded at room temperature for 6 h. Two isomers of the desired product were isolated by reversed-phase high-pressure liquid chromatography (RP-HPLC).

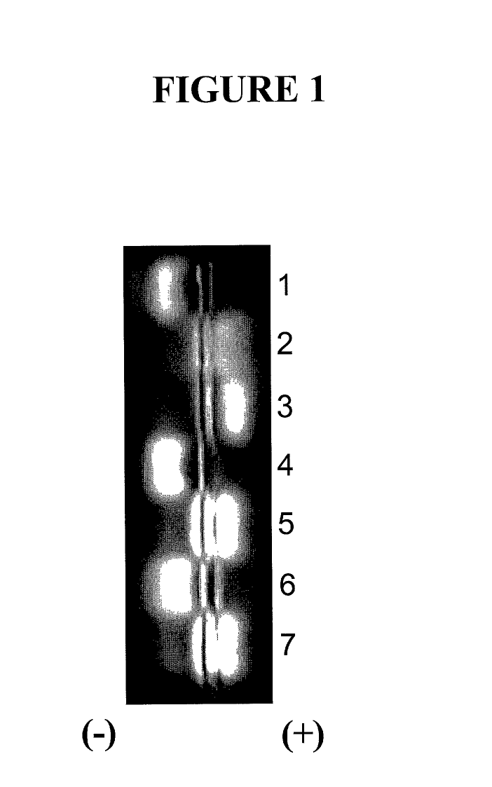

The fluorescent peptides were assayed with protein kinase A (PKA) using the PepTag assay kit from Promega (Madison, WI). The concentrations of the peptides in the assay were 60 μM, and the concentration of ATP was 1 mM. After completion of the assay the 25 μL reaction mixtures were spiked with 5 μL of 50% glycerol and submitted to gel electrophoresis in a 0.8% horizontal agarose slab in 50 mM TRIS HCl, pH 8.0. The gels were imaged with a fluorescence imager. The results appear in FIG. 1. The Lissamine Kemptide reaction mixtures (− & + kinase) are included as controls. It is clear that both isomers of N-(TXR)LRRASLG [SEQ. ID NO. 11] are substrates for PKA.

### Example 2

### Preparation and Characterization of Fluorescent Jeffamine₉₀₀ Derivative Peptide Substrates

An active-ester derivative of BODIPY TRX-SE was conjugated to the PEG by reacting with α,ω-diamino PEG (Jeffamine ED-900). The reaction proceeded at room temperature in acetonitrile at a ratio of ten moles of Jeffamine ED-900 per mole of fluorophore. After the fluorophore active ester was consumed completely, twenty equivalents of N-succinimidyl bromoacetate per equivalent of amine was added to the reaction mixture, according to Scheme 1, below:

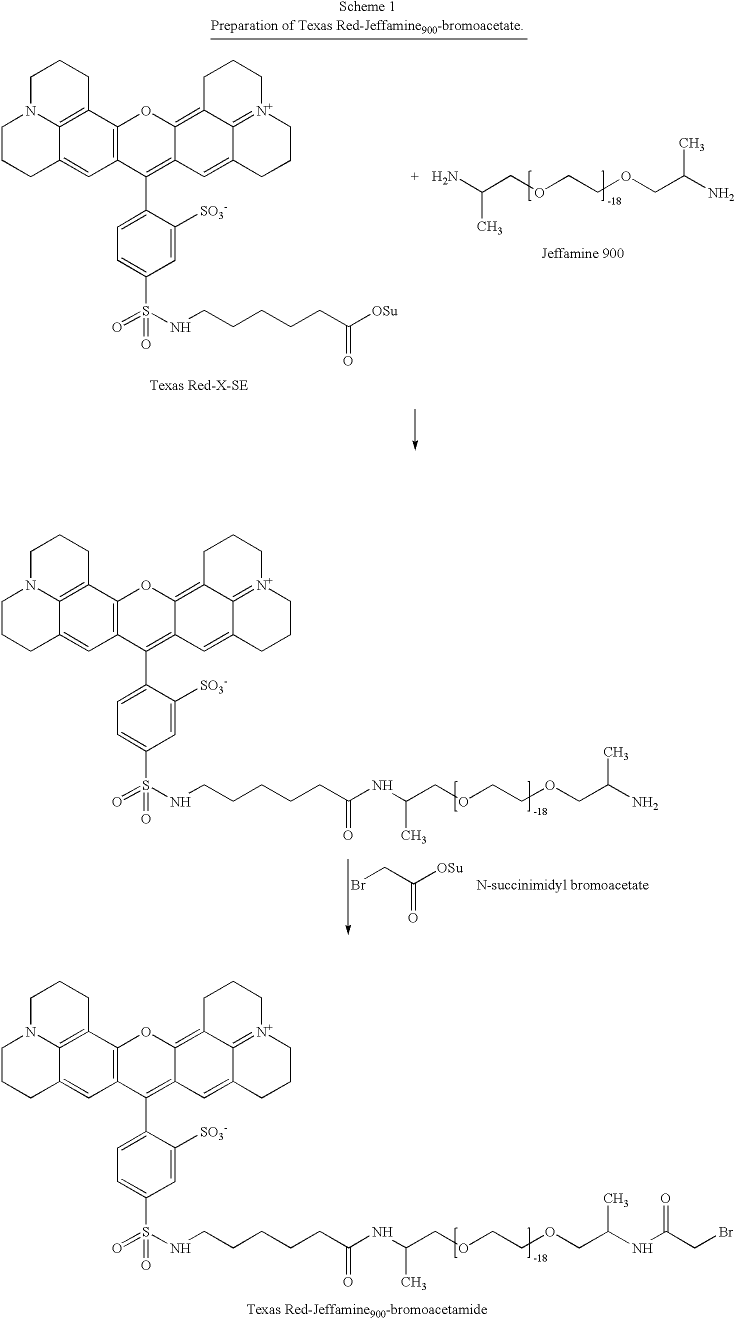

After the reaction was completed, the fluorophore-PEG-bromoacetamide was purified by liquid chromatography. The thiol-containing peptide CEEEFIYGAFKKKK [SEQ. ID NO. 8] was subsequently treated with the fluorophore-PEG-bromoacetamide to produce the fluorophore-PEG-peptidic substrate, as shown in scheme 2, below. This product was also purified by liquid chromatography.

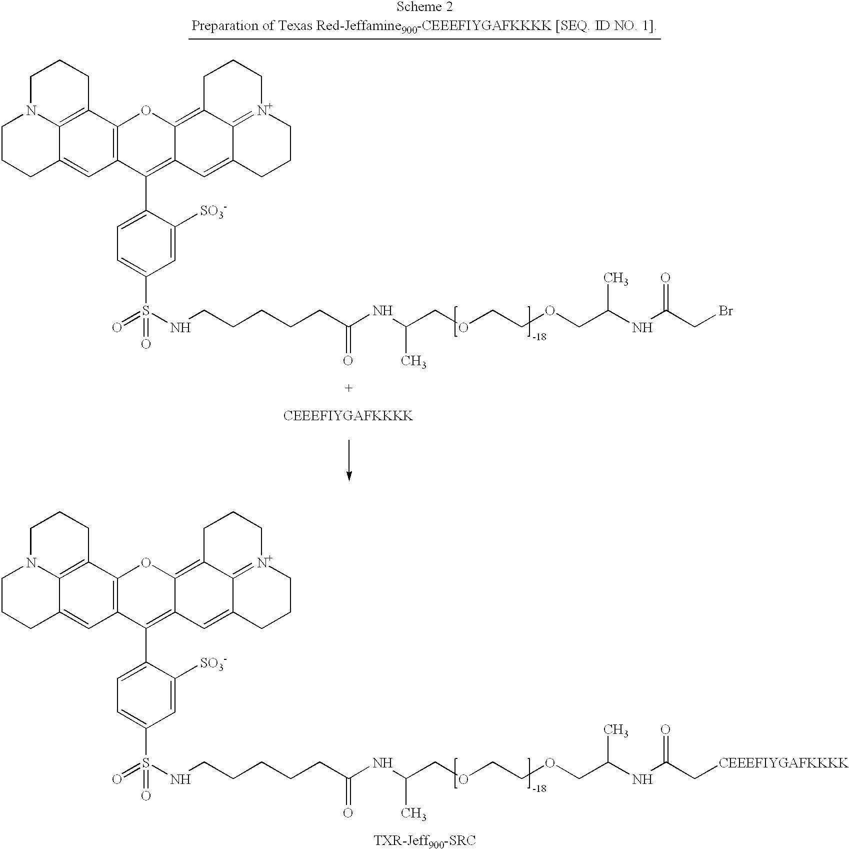

An assay of the fluorescent peptidic substrate was performed in the presence of SRC Kinase. The peptide concentration was 10 μM, and the ATP concentration was 100 μM. The reaction proceeded in a buffer comprising 25 mM sodium (N-[2-hydroxyethyl]piperazine-N′-[2-ethanesulfonate]), 20 mM magnesium chloride, pH 7.4 for 90 min at room temperature, and the reaction mixture was submitted to gel electrophoresis in a 0.8% agarose slab at pH 8.0. Nearly 75% conversion of the substrate to product took place, and the substrate and product migrated in opposite directions. The results appear in FIG. 3.

### Example 3

### Preparation and Characterization of Fluorescent PEG₃₄₀₀ Derivative Peptide Substrates

In another example the fluorophore BODIPY_{630/650} was coupled to the synthetic peptide LRRASLG [SEQ. ID NO. 10] (Kemptide) employing a 3,400 molecular weight PEG spacer. When a conjugate comprising just the BODIPY_{630/650} and Kemptide was made, its solubility was so low that Protein Kinase A could not phosphorylate it.

Because Kemptide already possesses good solubility and charge characteristics, and because the n-terminal lysine is useful for coupling reactions, no further modification of the peptide sequence was necessary. The PEG₃₄₀₀-BODIPY_{630/650} conjugate was prepared from H₂N-PEG₃₄₀₀-CO₂H (Shearwater Polymers, Inc.) and BODIPY_{630/650} X-SE (Molecular Probes) in acetonitrile. The product was purified on reversed-phase HPLC. BODIPY_{630/650}-PEG-CO₂H was activated with three equivalents each of EDCI and NHS in 50 mM MES, pH 5.5 for one hour. Kemptide was then added in 50 mM Na₂HPO₄, pH 9.5 to a final pH of 7.5. The desired conjugate was isolated by cation-exchange chromatography.

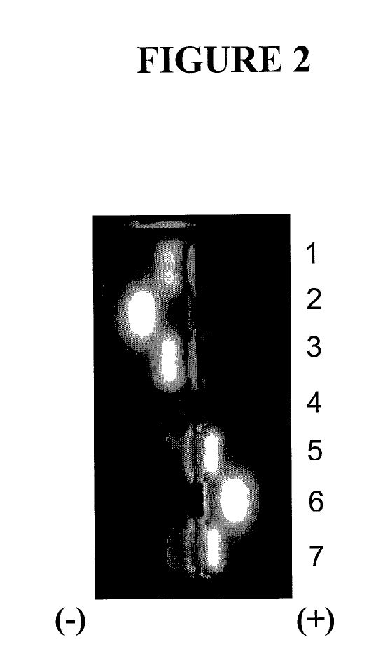

This fluorescent peptide PEG conjugate was successfully phosphorylated by PKA. The unphosphorylated and phosphorylated peptides migrate in an electric field as expected, albeit at a slower rate than the corresponding Lissamine-labeled peptides (FIG. 2). The slower migration may be due to sieving by the agarose gel, since the molecular length of the peptide is greatly increase by the incorporation of the PEG. Thus, as illustrated in this example, in addition to improving the hydrophilicity of the peptide substrates, the hydrophilic polymer linker may also be used to modify the apparent molecular weight and mobility of the synthetic substrates.

### Example 4

### Preparation of Fluorescent Jeffamine₉₀₀ Derivative Peptide Substrates

Another fluorescent SRC substrate was prepared with BODIPY TR-X, SE and Jeffamine ED-900 according to the procedure outlined in Scheme 1 and described in Example 2. To a stirring solution that comprised ten molar equivalents of Jeffamine ED-900 in dry acetonitrile was added drop wise one molar equivalent of BODIPY TR-X, SE in dry acetonitrile. The reaction proceeded at room temperature for several hours with stirring after which time twenty molar equivalents of solid N-succinimidyl bromoacetate was added. The reaction proceeded over night at room temperature. The desired product BODIPY TR-Jeffamine-bromoacetamide (BTR-Jeff-BAA) was purified by RP-HPLC. The SRC substrate CEEEFIYGAFKKKK [SEQ. ID NO. 8] was reacted with an excess of BTR-Jeff-BAA in an aqueous buffer consisting of 100 mM sodium phosphate, 1 mM EDTA pH 7.0 as outlined in Scheme 2 and described in Example 2. The reaction proceeded at room temperature for two hours. The product was isolated by RP-HPLC.

### Example 5

### Preparation and Characterization of Fluorescent Jeffamine₉₀₀ Derivative Peptide Substrates with N-Terminal Blocking Groups

Two potential SRC substrates that contain blocked amino termini were prepared using BTR-Jeff-BAA and the peptides Ac-CEEFIYGAFKKKK [SEQ. ID NO. 9] and Ac-CEEFIYGAFRRRR [SEQ. ID NO. 12]. The peptides were treated with an excess of TXR-Jeff-BAA in a buffer that consisted of 50 mM HEPES, 1 mM EDTA pH 7.5. The reactions proceeded at room temperature over night. The products were purified by RP-HPLC.

A kinase assay was performed with each of these peptides as substrates. The concentrations of the Ac-C-[S-(BTR-Jeff)]EEFIYGAFKKKK [SEQ. ID NO. 3] and Ac-C-[S-(BTR-Jeff)]EEFIYGAFRRRR [SEQ. ID NO. 4] were 20 μM, respectively. The ATP concentration in each reaction was 100 μM, and 10 units of SRC kinase were added per reaction. The reactions proceeded at room temperature for 1 h. The reaction mixtures were spiked with 50% glycerol to give a final concentration of 10%. The samples (25 μL) were submitted to gel electrophoresis on a 0.8% agarose slab in 50 mM TRIS HCl, pH 8.0.

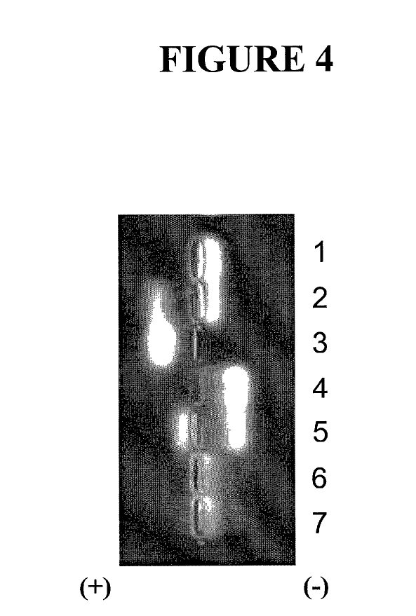

The results appear in FIG. 4. The anode is to the left and the cathode is to the right. C-[S-(TXR-Jeff)]EEEFIYGAFKKKK [SEQ. ID NO. 1] was included as a positive control. It is clear that Ac-C-[S-(BTR-Jeff)]EEFIYGAFKKKK [SEQ. ID NO. 3] is a substrate of SRC kinase, but Ac-C-[S-(BTR-Jeff)]EEFIYGAFRRRR [SEQ. ID NO. 4] is not. The solubility of Ac-C-[S-(BTR-Jeff)]EEFIYGAFRRRR [SEQ. ID NO. 4] is much lower than Ac-C-[S-(BTR-Jeff)]EEFIYGAFKKKK [SEQ. ID NO. 3], which may explain its poor mobility as well as lack of activity.

### Example 6

### Preparation and Characterization of Fluorescent Jeffamine₉₀₀ Derivative Peptide Substrates with N-Terminal Blocking Groups

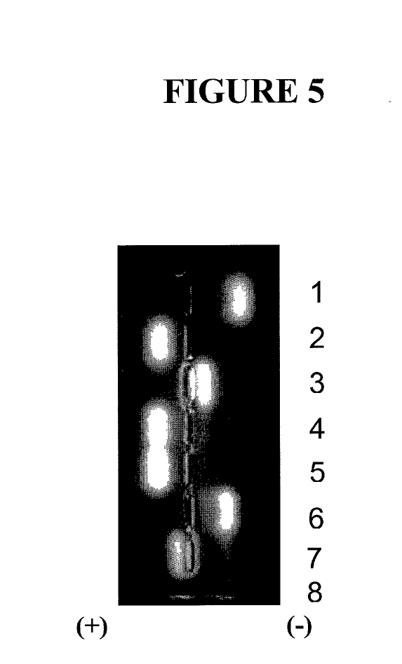

The SRC substrate Ac-C-[S-(TXR-Jeff)]EEFIYGAFKKKK [SEQ. ID NO. 5] was prepared and assayed as described by the method in Example 5 with the exceptions that TXR-Jeffamine-BAA was used as the fluorophore and the assay was allowed to proceed overnight. The results of the assay appear in FIG. 5. The anode is to the left and the cathode to the right. N-(TXR)-LRRASLG [SEQ. ID NO. 11] (Texas Red labeled Kemptide) and its phosphorylated derivative were included as mobility standards. Note that blockage of the amino terminus with an acetyl group resulted in an increased mobility of the substrate, but the product has decreased mobility as compared to the respective unacetylated peptides.

### Example 7

### Preparation and of Fluorescent Derivative Peptide Substrates with Designed Peptide Sequences and Comparison Fluorescent Jeffamine₉₀₀ Derivative Peptide Substrates

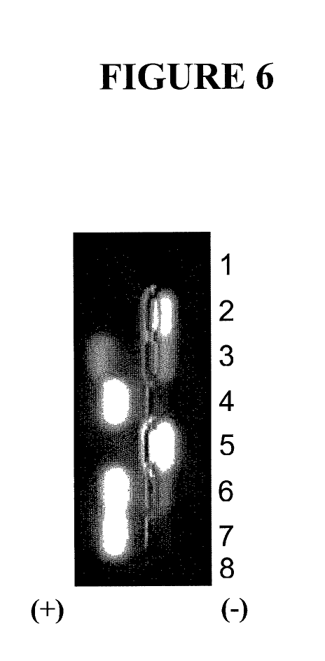

A fluorophore-labeled SRC substrate was prepared without the Jeffamine spacer. The SRC substrate CEEEFIYGAFKKKK [SEQ. ID NO. 8] (0.8 mg) was dissolved in 50 uL of 50 mM HEPES, 1 mM EDTA, pH 7.5 and to this solution was added a solution of Texas Red C₅ bromoacetamide (0.5 mg) in dry acetonitrile (50 uL). The reaction proceeded over night at room temperature. The precipitated product was dissolved in 50% aqueous acetic acid and purified by RP-HPLC. The purified peptide was assayed with SRC kinase as described in Example 5. The results of the assay appear in FIG. 6.

Sequence Listing

<110> Dwyer, Brian P.

We claim:

1. A water-soluble peptidic substrate with the general formula:
*F-R₁-L₁-R₂-P_{Hc1}-P_{S}-P_{Hc2}-(R₃-L₂-R₄-T)_{y}
wherein *F is a detectable moiety with a molecular weight of less than 5 kD;
R₁, R₂, R₃, and R₄ are each, independently: a covalent bond or a covalent linkage consisting of a branched or unbranched, substituted or unsubstituted, saturated or unsaturated chain of 1-10 carbon atoms; 0-3 heteroatoms selected from the group consisting of oxygen, nitrogen, and sulfur; and further consisting of at least one linkage chosen from the group consisting of ether, ester, hydrazone, amide, thioether, thioester, thiourea, disulfide and sulfonamide linkages;
L₁ and L₂ are each, independently: a branched or unbranched hydrophilic uncharged polymer selected from the group consisting of polyethylene glycol (PEG) and polysaccharides, and having a molecular weight of about 80 to about 4000 Daltons;
P_{Hc1} is peptide with the general formula A_{c}(A_{H})ₙAₘ,
wherein A_{c} is selected from the group consisting of a covalent bond, omithine, cysteine, homocysteine, cysteic acid, and lysine;
each of A_{H} is, independently, a charged or uncharged hydrophilic amino acid selected form the group consisting of serine, threonine, lysine, arginine, histidine, aspartic acid, glutamic acid, and cysteic acid;
n is an integer from 0 to 10;
Aₘ is selected from the group consisting of a covalent bond and methionine;
P_{Hc2} is a peptide with the general formula Aₘ(A_{H})ₙA_{c},
wherein A_{c}, if y is 1, is selected from the group consisting of a covalent bond, omithine, cysteine, homocysteine, cysteic acid, and lysine; or, if y is 0, is a terminating group selected from the group consisting of alcohol moieties, amine moieties, ester moieties, ether moieties, carboxylic acid moieties, amide moieties, and sulfonic acid moieties;
each of A_{H} is, independently, a charged or uncharged hydrophilic amino acid selected from the group consisting of serine, threonine, lysine, arginine, histidine, aspartic acid, glutamic acid, and cysteic acid;
n is an integer from 0 to 10;
Aₘ, is selected from the group consisting of a covalent bond and methionine;
P_{S} is a peptide from 5 to 25 amino acids in length;
T is a terminating group selected from the group consisting of alcohol moieties, amine moieties, ester moieties, ether moieties, carboxylic acid moieties, amide moieties, sulfonic acid moieties, quencher moieties, and detectable moieties; and
y is 0 or 1.

2. The substrate of claim 1 wherein P_{S} comprises a known protein-kinase recognition sequence.

3. The substrate of claim 1 wherein P_{S} comprises a known protein-phosphatase recognition sequence.

4. The substrate of claim 1 wherein P_{S} comprises a known protease recognition sequence.

5. The substrate of claim 1 wherein P_{S} comprises a phosphorylated amino acid residue selected from the group consisting of phosphoserine, phosphothreonine, and phosphotyrosine.

6. The substrate of claim 1 wherein R₂ is attached to the N-terminus of the peptidic portion of the molecule.

7. The substrate of claim 1 wherein R₂ is attached to the C-terminus of the peptidic portion of the molecule.

8. The substrate of claim 1 wherein the non-peptidic portion of the molecule is uncharged.

9. The substrate of claim 8 wherein the peptidic portion of the molecule carries a net positive charge.

10. The substrate of claim 8 wherein the peptidic portion of the molecule carries a net negative charge.

11. The substrate of claim 8 wherein the peptidic portion of the molecule is carries no net charge.

12. The substrate of claim 1 wherein *F is selected from the group consisting of a fluorescent moiety, a chromogenic moiety, and a chemiluminescent moiety.

13. The substrate of claim 1 wherein *F is a fluorescent moiety.

14. The substrate of claim 13 wherein the fluorescent moiety is selected from the group consisting of BODIPY_{630/650} X-SE, Texas Red X-SE, BODIPY TRX-SE, Cy-dyes, Lissamine, fluorescein, rhodamine, phycoerythrin, and coumarin.

15. The substrate of claim 1 wherein at least one of L₁ or L₂ is polyethylene glycol.

16. The substrate of claim 1 wherein at least one of L₁ or L₂ is a polysaccharide.

17. The substrate of claim 1 wherein at least one of L₁ or L₂ has a molecular weight of from about 100 to about 2000 Daltons.

18. The substrate of claim 1 wherein at least one of L₁ or L₂ has a molecular weight of from about 500 to about 1500 Daltons.

19. The substrate of claim 1 wherein at least one of L₁ or L₂ has a molecular weight of from about 800 to about 1000 Daltons.

20. The substrate of claim 1 wherein at least one of L₁ or L₂ is a polyethylene glycol having a molecular weight from about 230 to about 2000 Daltons.

21. The substrate of claim 1 wherein R₂ comprises an amide linkage.

22. The substrate of claim 1 wherein R₂ comprises a thiol linkage.

23. The substrate of claim 1 wherein for both P_{Hc1} and P_{Hc2}, A_{c} is a covalent bond and n is 0.

24. The substrate of claim 1 wherein for at least one of P_{Hc1} and P_{Hc2}, A_{c} comprises homocysteine.

25. The substrate of claim 1 wherein for at least one of P_{Hc1} and P_{Hc2}, A_{c} comprises cysteine.

26. The substrate of claim 1 wherein P_{Hc1} has a different net charge than P_{Hc2}.

27. The substrate of claim 1 wherein P_{Hc1} has a negative net charge and P_{Hc2} has a positive net charge.

28. The substrate of claim 1 wherein P_{Hc1} has a positive net charge and P_{Hc2} has a negative net charge.

29. The substrate of claim 1 wherein P_{S} is from 5 to 10 amino acids in length.

30. The substrate of claim 1 wherein P_{S} comprises a random amino acid sequence.

31. The substrate of claim 1 wherein P_{S} comprises a weighted random amino acid sequence.

32. The substrate of claim 1 wherein P_{S} comprises a partially random amino acid sequence.

33. The substrate of claim 1 wherein P_{S} comprises a sequence selected from a known enzyme substrate.

34. The substrate of claim 1 wherein y is 0.

35. The substrate of claim 1 wherein y is 1.

36. The substrate of claim 1 wherein T is a terminating moiety selected from the group consisting of alcohol moieties, amine moieties, ester moieties, ether moieties, carboxylic acid moieties, amide moieties, and sulfonic acid moieties.

37. The substrate of claim 1 wherein T is a quencher moiety.

38. The substrate of claim 1 wherein T is a detectable moiety selected from the group consisting of a fluorescent moiety, a chromogenic moiety, and a chemiluminescent moiety.

39. The substrate of claim 1 wherein T is a fluorescent moiety different from *F.

40. The substrate of claim 39 wherein T is selected from the group consisting of BODIPY_{630/650} X-SE, Texas Red X-SE, BODIPY TRX-SE, Cy-dyes, lissamine, fluorescein, rhodamine, phycoerythrin, and coumarin.

41. A library consisting of a plurality of water-soluble peptidic substrates, wherein each peptidic substrate member of the library has the general formula:
*F-R₁-L₁-R₂-P_{Hc1}-P_{S}-P_{Hc2}-(R₃-L₂-R₄-T)_{y}
wherein *F is a detectable moiety with a molecular weight of less than 5 kD;
R₁, R₂, R₃, and R₄ are each, independently: a covalent bond or a covalent linkage consisting of a branched or unbranched, substituted or unsubstituted, saturated or unsaturated chain of 1-10 carbon atoms; 0-3 heteroatoms selected from the group consisting of oxygen, nitrogen, and sulfur; and
further consisting of at least one linkage chosen from the group consisting of ether, ester, hydrazone, amide, thioether, thioester, thiourea, disulfide and sulfonamide linkages;
L₁ and L₂ are each, independently: a branched or unbranched hydrophilic uncharged polymer selected from the group consisting of polyethylene glycol (PEG) and polysaccharides, and having a molecular weight of about 80 to about 4000 Daltons;
P_{Hc1} is peptide with the general formula A_{c}(A_{H})ₙAₘ,
wherein A_{c} is selected from the group consisting of a covalent bond, omithine, cysteine, homocysteine, cysteic acid, and lysine;
each of A_{H} is, independently, a charged or uncharged hydrophilic amino acid selected form the group consisting of serine, threonine, lysine, arginine, histidine, aspartic acid, glutamic acid, and cysteic acid;
n is an integer from 0 to 10;
Aₘ is selected from the group consisting of a covalent bond and methionine;
P_{Hc2} is a peptide with the general formula Aₘ(A_{H})ₙA_{c},
wherein A_{c}, if y is 1, is selected from the group consisting of a covalent bond, omithine, cysteine, homocysteine, cysteic acid, and lysine; or, if y is 0, is a terminating group selected from the group consisting of alcohol moieties, amine moieties, ester moieties, ether moieties, carboxylic acid moieties, amide moieties, and sulfonic acid moieties;
each of A_{H} is, independently, a charged or uncharged hydrophilic amino acid selected from the group consisting of serine, threonine, lysine, arginine, histidine, aspartic acid, glutamic acid, and cysteic acid;
n is an integer from 0 to 10;
Aₘ, is selected from the group consisting of a covalent bond and methionine;
P_{S} is a peptide from 5 to 25 amino acids in length;
T is a terminating group selected from the group consisting of alcohol moieties, amine moieties, ester moieties, ether moieties, carboxylic acid moieties, amide moieties, sulfonic acid moieties, quencher moieties, and detectable moieties; and
y is 0 or 1.

42. The library of claim 41 wherein, for each member of the library, P_{S} comprises a phosphorylated amino acid residue selected from the group consisting of phosphoserine, phosphothreonine, and phosphotyrosine.

43. The library of claim 41 wherein, for each member of the library, R₂ is attached to the N-terminus of the peptidic portion of the molecule.

44. The library of claim 41 wherein, for each member of the library, R₂ is attached to the C-terminus of the peptidic portion of the molecule.

45. The library of claim 41 wherein, for each member of the library, the non-peptidic portion of the molecule is uncharged.

46. The library of claim 45 wherein, for each member of the library, the peptidic portion of the molecule carries a net positive charge.

47. The library of claim 45 wherein, for each member of the library, the peptidic portion of the molecule carries a net negative charge.

48. The library of claim 45 wherein, for each member of the library, the peptidic portion of the molecule is carries no net charge.

49. The library of claim 41 wherein, for each member of the library, *F is selected from the group consisting of a fluorescent moiety, a chromogenic moiety, and a chemiluminescent moiety.

50. The library of claim 41 wherein, for each member of the library, *F is a fluorescent moiety.

51. The library of claim 50 wherein the fluorescent moiety is selected from the group consisting of BODIPY_{630/650} X-SE, Texas Red X-SE, BODIPY TRX-SE, Cy-dyes, Lissamine, fluorescein, rhodamine, phycoerythrin, and coumarin.

52. The library of claim 41 wherein, for each member of the library, at least one of L₁ or L₂ is polyethylene glycol.

53. The library of claim 41 wherein, for each member of the library, at least one of L₁ or L₂ is a polysaccharide.

54. The library of claim 41 wherein, for each member of the library, at least one of L₁ or L₂ has a molecular weight of from about 100 to about 2000 Daltons.

55. The library of claim 41 wherein, for each member of the library, at least one of L₁ or L₂ has a molecular weight of from about 500 to about 1500 Daltons.

56. The library of claim 41 wherein, for each member of the library, at least one of L₁ or L₂ has a molecular weight of from about 800 to about 1000 Daltons.

57. The library of claim 41 wherein, for each member of the library, at least one of L₁ or L₂ is a polyethylene glycol having a molecular weight from about 230 to about 2000 Daltons.

58. The library of claim 41 wherein, for each member of the library, R₂ comprises an amide linkage.

59. The library of claim 41 wherein, for each member of the library, R₂ comprises a thioether linkage.

60. The library of claim 41 wherein, for each member of the library, for both P_{Hc1} and P_{Hc2}, A_{c} is a covalent bond and n is 0.

61. The library of claim 41 wherein, for each member of the library, for at least one of P_{Hc1} and P_{Hc2}, A_{c} comprises homocysteine.

62. The library of claim 41 wherein, for each member of the library, for at least one of P_{Hc1} and P_{Hc2}, A_{c} comprises cysteine.

63. The library of claim 41 wherein, for each member of the library, for at least one of P_{Hc1} and P_{Hc2}, A_{c} comprises methionine.

64. The library of claim 41 wherein, for each member of the library, P_{Hc1} has a different net charge than P_{Hc2}.

65. The library of claim 41 wherein, for each member of the library, P_{Hc1} has a negative net charge and P_{Hc2} has a positive net charge.

66. The library of claim 41 wherein, for each member of the library, P_{Hc1} has a positive net charge and P_{Hc2} has a negative net charge.

67. The library of claim 41 wherein, for each member of the library, P_{S} is from 5 to 10 amino acids in length.

68. The library of claim 41 wherein, for each member of the library, wherein P_{S} comprises a random amino acid sequence.

69. The library of claim 41 wherein, for each member of the library, P_{S} comprises a weighted random amino acid sequence.

70. The library of claim 41 wherein, for each member of the library, P_{S} comprises a partially random amino acid sequence.

71. The library of claim 41 wherein, for each member of the library, y is 0.

72. The library of claim 41 wherein, for each member of the library, y is 1.

73. The library of claim 41 wherein, for each member of the library, T is a terminating moiety selected from the group consisting of alcohol moieties, amine moieties, ester moieties, ether moieties, carboxylic acid moieties, amide moieties, and sulfonic acid moieties.

74. The library of claim 41 wherein, for each member of the library, T is a quencher moiety.

75. The library of claim 41 wherein, for each member of the library, T is a detectable moiety selected from the group consisting of a fluorescent moiety, a chromogenic moiety, and a chemiluminescent moiety.

76. The library of claim 41 wherein, for each member of the library, T is a fluorescent moiety different from *F.

77. The library of claim 76 wherein, for each member of the library, T is selected from the group consisting of BODIPY_{630/650} X-SE, Texas Red X-SE, BODIPY TRX-SE, Cy-dyes, lissamine, fluorescein, rhodamine, phycoerythrin, and coumarin.

78. A method of selecting peptidic substrates from the library of claim 41 for use in a protein-modifying enzyme assay, the method comprising:
(a) separating the members of the library which are soluble under suitable reaction conditions for the protein-modifying enzyme from those which are not soluble under suitable reaction conditions for the protein-modifying enzyme;
(b) combining the soluble members of the library obtained in (a) with the protein-modifying enzyme under suitable reaction conditions for the protein-modifying enzyme, thereby modifying some members of the library;
(c) separating the modified members of the library produced in (b) from the unmodified members of the library;
(d) determining the sequence of P_{S} for the modified members of the library.

79. The method of claim 78 wherein the protein-modifying enzyme is a protein-kinase, and the modification of the modified members of the library is the phosphorylation of a serine, threonine, or tyrosine amino acid residue.

80. The method of claim 78 wherein at least a portion of the members of the peptidic substrate library contain a phosphorylated amino acid residue selected from the group consisting of phosphoserine, phosphothreonine, and phosphotyrosine, and wherein the protein-modifying enzyme is a protein-phosphatase, and the modification of the modified members of the library is the dephosphorylation of a phosphoserine, phosphothreonine, or phosphotyrosine amino acid residue.

81. The method of claim 78 wherein the protein-modifying enzyme is a protease, and the modification of the modified members of the library is the cleavage of the peptidic portion of the modified members.

82. The method of claim 78 wherein the separation in (a) is by solvent phase partitioning between an organic solvent and an aqueous buffer suitable for use with the protein-modifying enzyme.

83. The method of claim 78 wherein the separation in (c) is by metal chelation chromatography.

84. The method of claim 83 wherein the metal chelation chromatography is carried out on a column containing a chelated cation selected from the group consisting of Fe⁺³ and Ga⁺³.

85. The method of claim 78 wherein the separation in (c) is by chromatofocusing chromatography on an anion exchange column.

86. The method of claim 78 wherein the separation in (c) is by electrophoretic separation of the modified and unmodified members of the library.

87. The method of claim 78 wherein R₂ is attached to the N-terminal end of the peptide portion of the peptidic substrates, and wherein the sequence determination in (d) is by C-terminal degradation of the peptidic portion of the modified members of the library.

88. The method of claim 78 wherein the sequence determination in (d) is by Edman degradation of the peptidic portion of the modified members of the library.

89. The method of claim 88 wherein R₂ is attached to the C-terminal end of the peptide portion of the peptidic substrates.

90. The method of claim 88 wherein R₂ is attached to the N-terminal end of the peptide portion of the peptidic substrates, and further comprising the step of cleaving the peptide portion of the peptidic substrates from the labeled hydrophilic polymer linker portion of the peptidic substrates.

91. The method of claim 90 wherein, for the members of the peptidic libraries, A, in P_{Hc1} comprises methionine, and the cleavage is by cyanogen bromide cleavage of the substrates at the methionine residue.

92. A method of assaying a molecule of interest for its effect on a protein-kinase or protein-phosphatase reaction, the method comprising:
(a) combining the molecule of interest, an enzyme selected from the group consisting of protein-kinases and protein-phosphatases, and one or more peptidic substrates of claim 1, wherein a Ps comprising a recognition sequence for the protein kinase is within one or more of the peptidic substrates, under conditions suitable for the activity of the enzyme;
(b) terminating the activity of the enzyme after a period of time;
(c) electrophoretically separating the phosphorylated peptidic substrate from the unphosphorylated peptidic substrate to produce a localized phosphorylated peptidic substrate fraction and unphosphorylated peptidic substrate fraction;
(d) quantifying at least one of the separated fractions by detecting a detectable moiety on the peptidic substrate in the localized fraction, thereby determining the extent of conversion of the substrate by the enzyme during the period of time.

93. The method of claim 92, the method further comprising a step (e) comparing the extent of conversion of the substrate by the enzyme in step (d) with the extent of conversion by the enzyme when the enzyme is combined with the peptidic substrate under conditions suitable for the action of the enzyme for a substantially identical period of time in the absence of the molecule of interest.

94. The method of claim 92, the method further comprising a step (e) comparing the extent of conversion of the substrate by the enzyme in step (d) with the extent of conversion by the enzyme when the enzyme is combined with the peptidic substrate under conditions suitable for the action of the enzyme for a substantially identical period of time in the absence of the molecule of interest and in the presence of a molecule of known effect on the enzyme.

95. The method of claim 92 wherein the enzyme is a protein-kinase.

96. The method of claim 92 wherein the enzyme is a protein-phosphatase, and the peptidic substrates are phosphorylated in step (a).

97. The method of claim 92 wherein the period of time is in the range of 15 minutes to 2 hours.

98. The method of claim 92 wherein the period of time is in the range of 2 to 4 hours.

99. The method of claim 92 wherein the period of time is in the range of 4 to 8 hours.

100. The method of claim 92 wherein the period of time is in the range of 8 hours to 48 hours.

101. The method of claim 92 wherein the unphosphorylated peptidic substrate carries a net positive charge, and the phosphorylated peptidic substrate carries a net negative charge

102. The method of claim 92 wherein the unphosphorylated peptidic substrate carries no net charge, and the phosphorylated peptidic substrate carries a net negative charge.

103. The method of claim 92 wherein the unphosphorylated peptidic substrate carries a net positive charge, and the phosphorylated peptidic substrate carries no net charge.

104. The method of claim 92 wherein the unphosphorylated peptidic substrate carries a net negative charge, and the phosphorylated peptidic substrate carries a net negative charge.

105. The method of claim 92 wherein *F is a fluorescent moiety, and the detecting in (c) is fluorometric detection.

106. A method of assaying a molecule of interest for its effect on a protease reaction, the method comprising:
(a) combining the molecule of interest, a protease, and one or more peptidic substrates of claim 1, wherein a P_{S} comprising a recognition sequence for the protease is within one or more of the peptidic substrates, under conditions suitable for the activity of the protease
(b) terminating the activity of the protease after a period of time;
(c) electrophoretically separating the cleaved peptidic substrate from the uncleaved peptidic substrate to produce at least one localized cleaved peptidic substrate fraction and an uncleaved peptidic substrate fraction;
(d) quantifying at least one of the separated fractions by detecting a detectable moiety on the peptidic substrate in the localized fraction, thereby determining the extent of conversion of the substrate by the protease during the period of time.

107. The method of claim 106, the method further comprising a step (e) comparing the extent of conversion of the substrate by the protease in step (d) with the extent of conversion by the protease when the protease is combined with the peptidic substrate under conditions suitable for the action of the protease for a substantially identical period of time in the absence of the molecule of interest.

108. The method of claim 106, the method further comprising a step (e) comparing the extent of conversion of the substrate by the protease in step (d) with the extent of conversion by the protease when the protease is combined with the peptidic substrate under conditions suitable for the action of the protease for a substantially identical period of time in the absence of the molecule of interest and in the presence of a molecule of known effect on the enzyme.

109. The method of claim 106 wherein the uncleaved peptidic substrate carries a net positive charge, and the portion of the cleaved peptidic substrate comprising *F carries a net negative charge.

110. The method of claim 106 wherein the uncleaved peptidic substrate carries no net charge, and the portion of the cleaved peptidic substrate comprising *F carries a net negative charge

111. The method of claim 106 wherein the uncleaved peptidic substrate carries a net negative charge, and the portion of the cleaved peptidic substrate comprising *F carries a net positive charge.

112. The method of claim 106 wherein the uncleaved peptidic substrate carries no net charge, and the portion of the cleaved peptidic substrate comprising *F carries a net positive charge.

113. The method of claim 106 wherein *F is a fluorescent moiety, and the detecting in (c) is by fluorometric detection.
